(19) United States
(12) Patent Application Publication
(10) Pub. No.: US 2004/0181343 A1
(43) Pub. Date: Sep. 16, 2004

(54) Computer program products and systems for rapidly changing the solution environment around sensors

(75) Inventors: Joakim Wigstrom, Frolunda (SE); Jon Sinclair, Goteborg (SE)

Correspondence Address: EDWARDS & ANGELL, LLP, P.O. Box 9169, Boston, MA, 02209, US

(73) Assignee: Cellectricon AB

(21) Appl. No.: 10/698,599

(22) Filed: Oct. 31, 2003

Related U.S. Application Data
(60) Provisional application No. 60/423,197, filed on Nov. 1, 2002.

(51) Int. Cl. G06F 19/00; G01N 33/48; G01N 33/50

(52) U.S. Cl. 702/19; 702/20

(57) ABSTRACT

The invention provides computer program products for coordinating the movement of cells and other components in a microfluidic substrate with data acquisition. The microfluidic workstation may be used to examine the physiological responses of ion channels, receptors, neurons, and other cells to fluidic streams. The system may also be useful for screening compound libraries to search for novel classes of compounds, screening members of a given class of compounds for effects on specific ion channel proteins and receptors, and to rapidly determine dose-response curves in cell-based assays.

### RELATED APPLICATIONS

This application claims priority under 35 U.S.C. § 119(e) to U.S. Provisional Application Serial No. 60/423,197 filed Nov. 1, 2002, the entirety of which is incorporated by reference herein.

### FIELD OF THE INVENTION

The invention relates to computer program products and systems for performing high throughput screening (HTS) assays using microfluidic substrates.

### BACKGROUND OF THE INVENTION

Ion-microchannels are important therapeutic targets. Neuronal communication, heart function, and memory all critically rely upon the function of ligand-gated and voltage-gated ion channels. In addition, a broad range of chronic and acute pathophysiological states in many organs such as the heart, gastrointestinal tract, and brain involve ion channels. Indeed, many existing drugs bind receptors directly or indirectly connected to ion channels. For example, anti-psychotic drugs interact with receptors involved in dopaminergic, serotonergic, cholinergic, noradrenergic and glutamatergic neurotransmission.

Because of the importance of ion channels as drug targets, there is a need for methods which enable high throughput screening (HTS) of compounds acting on ligand-gated and voltage-gated channels. However, existing HTS drug discovery systems targeting ion channels generally miss significant drug activity because they employ indirect methods, such as raw binding assays or fluorescence-based readouts. Although as many as ten thousand drug leads can be identified from a screen of a million compounds, identification of false positives and false negatives can still result in a potential highly therapeutic blockbuster drug being ignored, and in unnecessary and costly investments in false drug leads.

Patch clamp methods are superior to any other technology for measuring ion microchannel activity in cells, and can measure currents across cell membranes in ranges as low as picoAmps (see, e.g., Neher and Sakmann, Nature 260: 799-802; Hamill, et al., 1981, Pflugers Arch 391: 85-100; Sakmann and Neher, 1983, In Single-Microchannel Recording pp. 37-52, Eds. B. Sakmann and E. Neher. New York and London, Plenum Press).

Attempts have been made to use patch-clamp recordings in HTS platforms. For example, Sörensen et al., in WO 96/13721, describe a system that couples an HPLC autosampler to a micro-flow chamber in which a patch-clamped cell is placed. While the system enables multiple compounds to be assayed at a time, the system creates large dead volumes and solution exchange is generally slow compared to activation times of ion channel receptors.

Another strategy to develop patch-clamp-based HTS systems involves microfabrication of a plurality of patch-clamp-electrodes on solid supports using microfabrication techniques. Klemic, et al., in WO 01/59447, describe one such planar patch clamp electrode array comprising a plurality of electrodes for performing patch clamp recordings on a plurality of patch-clamped cells. Samples of cells and solutions are provided to the array by pouring, immersing the electrodes, or pipetting into wells containing the cells and electrodes. However, using such planar surfaces it has been difficult to obtain stable recording configurations for prolonged periods of time with good electrical properties.

U.S. Published application Ser. No. 2002/0076689 describes an automated electrophysiology workstation for performing patch clamp analysis on Xenopus oocytes and associated instrumentation software, but does not make use of microfabricated substrates.

### SUMMARY OF THE INVENTION

In particular, the invention provides computer program products for coordinating the movement of cells and other components in a microfluidic substrate with data acquisition.

The microfluidic workstation may be used to examine the physiological responses of ion channels, receptors, neurons, and other cells to fluidic streams. The system may also be useful for screening compound libraries to search for novel classes of compounds, screening members of a given class of compounds for effects on specific ion channel proteins and receptors, and to rapidly determine dose-response curves in cell-based assays.

In one aspect, the invention provides a computer program product embedded in a computer readable medium, comprising instructions for controlling one or more functions of a microfluidic substrate in response to received data regarding one or more substrate properties. Preferably, at least one of the functions comprises scanning a sensor, such as a cell, relative to an outlet of at least one microchannel in the substrate. More preferably, the sensor is scanned relative to outlets of a plurality of microchannels. In another aspect, the computer program product provides instructions to expose the microfluidic substrate to a plurality of interdigitating fluid streams comprising alternating streams of agent and buffer.

The computer program product is generally embedded in a medium comprising a memory and data relating to substrate properties and or parameters of functions are stored in the memory. Exemplary substrate function parameters include: number of microchannel outlets to be scanned, time to complete scanning, length of pauses at microchannel outlets, and maximum speed of scanning, trajectory of scanning and the like. Stored data can be accessed to implement one or more substrate fictions. Substrate properties include, but are not limited to: number of microchannels in the substrate, microchannel geometry and distance between microchannel outlets. Substrate functions include, but are not limited to: fluid movement; separation; concentration; mixing; heating; focusing; and detection.

In one aspect, the computer program product further comprises instructions for generating and displaying a graphical user interface. The graphical user interface displays a screen on which one or more substrate properties is displayed, i.e., the number of microchannels, distance between microchannels, and microchannel geometry. In another aspect, the graphical user interface displays one of more properties of a macroscale device for interfacing with the microfluidic substrate, such as stage type, driver system, micropositioner type, stage port. Preferably, the graphical user interface comprises fields for inputting one or more function parameters, such as numbers of microchannel outlet to be scanned, time to complete scanning, length of pauses at microchannel outlets, trajectory of scanning, and maximum speed of scanning. In certain embodiments, where changes in pressure in at least one microchannel is used to scan (e.g., where a sensor is stationary), the interface comprises field for inputting pressure and or pressure changes (e.g., increase, decrease, etc). The graphical user interface can also provide selectable buttons, check boxes, and/or sliders, displaying values for one or more function parameters, e.g., number of microchannel outlet to be scanned, time to complete scanning, length of pauses at microchannel outlets, maximum speed of scanning, pressure at a microchannel and the like.

In one preferred aspect, the graphical user interface provides options for a plurality of different scan modes. Scanning may be performed using one or more of the scan modes and in one aspect, scanning is performed using a plurality of different scan modes. Scan modes include, but are not limited to, alternating channel delay, continuous movement, or an input trigger scan mode. Continuous scanning comprises sweeping the sensor across a plurality of microchannels without pausing. Alternating channel delay comprises scanning the sensor past a plurality of microchannels while including one or more pause intervals. Input trigger stepping comprises scanning in response to a selected threshold signal received from the substrate.

The invention further provides, a computer program product comprising a data acquisition program embedded in a computer readable medium, the data acquisition program, comprising: a search function; and a relationship determining function. The computer readable medium further comprises a memory comprising data relating to scanning a sensor across one or more fluid streams of a microfluidic device. In one aspect, data relating to scanning the sensor comprises data relating to the number of microchannel outlets scanned, the time to complete a scan, pause time intervals at one or more channels, a type of fluid stream delivered by one or more microchannel outlets, and data relating to the sensor response at one or more microchannel outlets. In another aspect, in response to data acquired, the data acquisitions system provides data to the external hardware to alter one or more substrate functions, either directly, or by providing the data to the application program, e.g., to repeat scanning across selected channels of the substrate or to otherwise alter scanning parameters.

In another aspect, the cell is a sensor and the sensor response comprises a change in electrical properties of the cell.

The invention also provides a microfluidic workstation comprising: a computer program product as discussed above operably linked to a microfluidic substrate. The computer program communicates with external system hardware coupled to one or more macroscale components interfaced with the microfluidic substrate. In response to this communication, one or more substrate functions, e.g., such as scanning of a sensor relative to the substrate, can be executed.

Preferably, the microfluidic workstation further comprises a data processing system comprising a memory. The data processing system accesses data from one or more of the computer program products. The data processing system accesses data relating to system properties or functions.

The external processing hardware transmits signals to the computer program products for controlling one or more substrate functions. In one aspect, the data processing device can access data received by the computer program for controlling substrate functions and provides the data to a data acquisition program. In one preferred aspect, the workstation further comprises a user device for displaying a graphical user interface. In another aspect, the workstation further comprises one or more amplifiers for patch clamp detection.

In a further preferred aspect, the microfluidic workstation further comprises a stage for receiving the substrate which can be scanned in one or more of an x-, y-, or z-direction and/or by rotating.

In still a further aspect, the external hardware of the workstation communicates with one or more macroscale components which interface with the microfluidic substrate. Exemplary macroscale components comprise a stage, an optical system, a detector, an amplifier, a fluid delivery system, a pump head, a pump, a separation device, a concentration device, and a micropositioner. Preferably, the micropositioner is for positioning a sensor. The micropositioner can comprise a micropipette, a nanopipette, a nanoelectrode, and a nanoelectrode array. One or more components, include but are not limited to: a valve, an electrically conducting element, a nanoelectrode, an electroporation mechanism; a sensor; and a heat conducting element.

The microfluidic workstation preferably includes a microfluidic substrate which comprises a sensor chamber into which the outlets of one or more microchannels open. The sensor chamber may comprise one or more sensors. In one aspect, the one or more sensors comprise one or more cells. In another aspect, the data acquisition program comprises a computer program product for patch clamp data acquisition and analysis.

Preferably, the workstation further comprises a data processing device wherein the data processing device can access data received by program for altering substrate functions and/or from a computer program product for patch clamp data acquisition and analysis.

The microfluidic workstation may further comprise one or more joysticks for controlling movement of a stage on which the substrate is placed. The joysticks can be used to locate and identify a first and last microchannel on the substrate.

Preferably, the microfluidic workstation comprises a graphical user interface in communication with the data processing device and displays a representation of the substrate on a screen of the user interface.

The invention further provides a suite of computer program products comprising one or more of the computer program products discussed above. Preferably, the suite comprises at least the program product for controlling substrate function and the data acquisition program.

The invention also provides a method for programmably exposing a sensor to a fluid stream. The method comprises providing a microfluidic substrate comprising a sensor chamber, and at least one microchannel opening into the sensor chamber at an outlet, the sensor chamber further comprising a sensor. Data is provided to a computer program product for controlling substrate function regarding one or more substrate properties and in response to this data, the computer program product provides instructions to external system hardware to execute one or more scanning functions such that the substrate, the sensor, or the substrate and the sensor move relative to one another, thereby scanning the sensor across the opening of the microchannel outlet. Preferably, the substrate comprises a plurality of microchannels with outlets opening into the sensor chamber and wherein the sensor is scanned across the plurality of outlets, thereby scanning the sensor across a plurality of fluid streams. Scanning may be continuous or the sensor may be paused at one or more channel outlets.

In one preferred aspect, at least one of the fluid streams comprises an agent.

In another preferred aspect, the fluid streams provide interdigitating fluid streams of agent and buffer and the sensor is sequentially scanned across the fluid streams.

In another aspect, the method comprises measuring a response of the sensor to one or more fluid streams, such as a change in an electrical property of the sensor. In a further aspect, the sensor is a cell and the response is measured by patch clamp analysis.

The invention additionally provides a method for scanning a sensor across one or more fluid streams delivered by one or more microchannels in a substrate, comprising the step of: entering data relating to the properties of the substrate into the display of an interface of user device in communication with a data processing system; wherein in response to the entering, the sensor is scanned across the one or more fluid streams.

### BRIEF DESCRIPTION OF THE FIGURES

The objects and features of the invention can be better understood with reference to the following detailed description and accompanying drawings.

FIG. 1A is an illustration of a microfluidic workstation according to one aspect of the invention. Double-headed arrows in the Figure refer to the two-way communication that occurs between various components of the system, illustrated as boxes in the Figure. In the embodiment shown in the Figure, the workstation comprises a microfluidic substrate whose position is scanned relative to a patch clamp pipette in response to instructions from an application program. The application program communicates with external system hardware, such as a controller operably linked to a stage on which the microfluidic substrate is placed and/or a controller operably linked to a patch clamp pipette positioner. The movement of the substrate relative to the pipette is controlled by movement of the stage and/or pipette. Data obtained by patch clamp is transmitted to a data acquisition program which may additionally comprise an analysis function. In response to this data, the analysis system may provide instructions to the external system hardware to change scanning parameters. In the embodiment shown in FIG. 1A, the application software and acquisition software communicate through a network (e.g., a WAN or LAN). FIG. 1B shows another aspect of the invention in which the acquisition program and application program (indicated as “dynaflow program”) are executed on different computers. Instructions from the dynaflow program are communicated to a stage controller which in turn controls the movement of a motorized stage on which a microfluidic substrate (“dynaflow chip”) is placed. However, the functions of application software and acquisition software may be executed through a single data processing system or computer. In the embodiment shown in FIG. 1B, an additional system function, fluid flow, is controlled by the application program which communicates with external hardware linked to the pump (indicated as “controller” in the Figure). An I/O card may be provided to facilitate sending and receiving triggers to and from the acquisition system. FIG. 1C is a schematic illustrating the various steps executed by a data processing system executing the dynaflow program. The diagram illustrates as examples how microfluidic substrate functions (scanning and fluid flow) can be controlled by the program. Parallel arrows going in opposite directions in FIG. 1B illustrate feedback between the various functions of the program and components of the microfluidic workstation. FIG. 1D is a schematic illustrating various operations of the microfluidic system.

FIG. 2 is a portion of a screen shot illustrating a portion of a graphical user interface for receiving information regarding the properties of a microfluidic substrate.

FIG. 3 is a portion of a screen shot illustrating a portion of a graphical user interface for configuring stage settings for scanning a sensor (e.g., such as a cell) relative to a microfluidic substrate.

FIG. 4 is a portion of a screen shot illustrating a portion of a graphical user interface for selecting output trigger settings for controlling one or more actions on a microfluidic substrate.

FIG. 5 is a portion of a screen shot illustrating a portion of a graphical user interface for selecting input trigger settings for synchronizing the movement of a sensor, relative to a microfluidic substrate with external data acquisition hardware.

FIG. 6A is a portion of a screen shot illustrating a portion of a graphical user interface which indicates the status of a stage for scanning a microfluidic substrate. FIG. 6B is a portion of a screen shot illustrating a portion of a graphical user interface for identifying substrate properties of the microfluidic substrate, e.g., such as the coordinates of the first and last microchannel on the substrate.

FIG. 7 is a screen shot of a graphical user interface comprising the sections shown in FIGS. 1, 2, 6A and 6B.

FIG. 8 is a screen shot of a graphical user interface comprising the sections shown in FIGS. 3, 4, and 5.

FIGS. 9A-C show top views of different embodiments of microfluidic chips according to aspects of the invention illustrating exemplary placements of reservoirs for interfacing with 96-well plates. FIG. 9A shows a chip comprising ligand reservoirs (e.g., the reservoirs receive samples of ligands from a 96-well plate). FIG. 9B shows a chip comprising alternating or interdigitating ligand and buffer reservoirs (e.g., every other reservoir receives samples of ligands from one 96-well plate, while the remaining reservoirs receive samples of buffer from another 96-well plate). As shown in FIG. 9C, additional reservoirs can be placed on chip for the storage and transfer of cells or other samples of interest.

FIG. 10A is a perspective view of a 3D chip design according to one aspect of the invention, in which the chip comprises a bottom set and top set of channels. FIG. 10B is a side view of FIG. 10A, showing fluid flow can be controlled through pressure differentials so that fluid flowing out of a channel in the bottom set will make a “U-turn” into an overlying channel. FIG. 10C is a top view of FIG. 10A and shows cell scanning across the “U-turn” fluid streams.

FIGS. 11A-N are schematics showing chip designs for carrying out cell scanning across ligand streams using buffer superfusion to provide a periodically resensitized sensor. FIG. 11A is a perspective view of the overall chip design and microfluidic system. FIGS. 11B-G show enlarged views of the outlets of microchannels and their positions with respect to a superfusion capillary and a patch clamp pipette, as well as a procedure for carrying out cell superfusion while scanning a patch-clamped cell across different fluid streams. “P” indicates a source of pressure on fluid in a microchannel or capillary. Bold arrows indicate direction of movement. FIGS. 11H-11N show a different embodiment for superfusing cells. As shown in the perspective view in FIG. 11H, instead of providing capillaries for delivering buffer, a number of small microchannels placed at each of the outlets of the ligand delivery channels are used for buffer delivery. As a patch-clamped cell is moved to a ligand channel and the system detects a response, a pulse of buffer can be delivered via the small microchannels onto the cell for superfusion. The advantage to using this system is that the exposure time of the patch-clamped cell to a ligand can be precisely controlled by varying the delay time between signal detection and buffer superfusion. FIG. 11I is a cross-section through the side of a microfluidic system used in this way showing proximity of a patch-clamped cell to both ligand and buffer outlets. FIG. 11J is a cross section, front view of the system, showing flow of buffer streams. FIG. 11K is a cross-section through a top view of the device showing flow of ligand streams and placement of the buffer microchannels. FIGS. 11L-11M show use of pressure applied to a ligand and/or buffer channel to expose a patch clamped cell to ligand and then buffer.

FIG. 12A schematically depicts a top view of the interdigitating channels of a microfluidic chip, with a patch-clamped cell being moved past the outlets of the channels. FIGS. 12B and 12C depict side views of alternate embodiments of the outlets and microchannels. FIGS. 12B and 12C are side views showing a 2D and 3D microfluidic chip design, respectively.

FIGS. 13A-C show agonist screening according to one method of the invention using a microfluidic chip comprising 26 outlets feeding into a sensor chamber. As shown in FIG. 13A, the screen is performed linearly from channel outlet position 1 to 26. The scans can be repeated until a sufficient number of scans are performed. A simulated trace and score sheet are shown in FIGS. 13B and C for a single forward scan across microfluidic channel outlets. From this analysis, α 6 is the agonist with highest potency, followed by α 2.

FIGS. 14A-C show a method for agonist screening using a microfluidic chip comprising 14 outlets feeding into a sensor chamber and high repetition rate buffer superfusion using a fluidic channel placed close to a patch-clamped cell. As shown in FIG. 14A, the screen is performed linearly from channel outlet position 1 to-14. The scans can be repeated until a sufficient number of scans are performed. A simulated trace for a single forward scan across microfluidic channel outlets and score sheet are shown in FIGS. 14B-C. A plurality of peak responses are obtained per single microchannel outlet. From this analysis, α 3 is the agonist with highest potency, followed by α 5.

FIGS. 15A-C show a method for dose-response screening using a microfluidic chip comprising 56 outlets feeding into a sensor chamber. As shown in FIG. 15A, the screen is performed linearly from channel outlet position 1 to 56. The scans can be repeated until a sufficient number of scans are performed. A simulated trace and score sheet are shown in FIGS. 15B and C for a single forward scan across microfluidic channel outlets varying doses across channels 1-28 (FIG. 15C). From these data, a dose-response curve can be created for the unknown agonist \tilde{α}.

FIGS. 16A-C show a method for antagonist screening according to one aspect of the invention using a microfluidic chip comprising 26 outlets feeding into a sensor chamber. As shown in FIG. 16A, the screen is performed linearly from position 1-to-26. The scans can be repeated until a sufficient number of scans are performed. As shown in the simulated trace and score sheet, FIGS. 16B and C, respectively, for a single forward scan across microfluidic channel outlets, □3 is the antagonist with highest potency followed by □5.

FIG. 17 shows whole cell patch clamp recordings of transmembrane current responses elicited by manual repeated scanning of a cell across the channel outlet where it was superfused by buffer into an open reservoir containing acetylcholine (1 mM). A train of peaks are produced by repeated manual scanning of the patched cell across the superfusion-generated gradient. The cell was scanned back and forth at an average scan rate of 100 μm/s and at a maximum rate of up to 150 μm/s across the entire outlet of the microchannel depicted in the inset.

FIGS. 18A-D show patch clamp current responses of a whole cell to 1 mM acetylcholine as the patch-clamped cell is scanned across the outlets of a parallel 7-channel structure (same structure as that shown in FIG. 19B). Channels 1, 3, 5 and 7 were filled with PBS buffer, while channels 2, 4 and 6 were filled with acetylcholine. The channel flow rate was 6.8 mm/s and the cell scanning speeds in the Figures were A) 0.61 nun/s, B) 1.22 mm/s, C) 2 mm/s and in D) 4 mm/s.

FIGS. 19A and B are microphotographs showing flow profiles at the outlet of a single microchannel (FIG. 19A) and an array of microchannels (FIG. 19B). Fluid flow was imaged under fluorescence using a fluorescent dye (fluorescein) as a flow tracer. The channels were 100μm wide, 50 μm thick, with an inter-channel spacing of 25 μm; the flow rate was 4 mm/s.

FIG. 20 shows patch clamp current responses of a whole cell to 1 mM acetylcholine as the patch-clamped cell was scanned across the outlets of a 7-channel structure (not shown) Channels 1, 3, 5 and 7 were filled with PBS buffer; channels 2, 4 and 6 with acetylcholine. The channel flow rate was 2.7 mm/s and the cell scanning speed was 6.25 μm/s.

FIG. 21 shows concentration-dependent patch clamp current responses of whole cells to 1 μM, 12 μM and 200 μM nicotine as the patch-clamped cell was scanned across the outlets of a 7-channel structure (not shown); channels 1, 3, 5 and 7 were filled with PBS buffer; channel 2 with 1 μM, 4 with 12 μM and 6 with 200 μM nicotine respectively. The flow rate was 3.24 mm/s and the cell scanning speed was 250 μm/s.

### DETAILED DESCRIPTION

The invention provides an automated workstation for controlling various processes in a microfluidic substrate and for controlling the movement of one or more sensors relative to such a substrate. The invention further provides computer program products for integrating functions and movements in a microfluidic substrate and for coordinating such fictions and movements with data acquisition.

Definitions

All technical and scientific terms used herein have the same meaning as commonly understood by one of ordinary skill in the art to which this invention belongs, unless defined otherwise. To facilitate the understanding of this invention, a number of terms are defined below. Terms defined herein have meanings as commonly understood by a person of ordinary skill in the areas relevant to the present invention.

Terms such as “a,” “an,” and “the” are not intended to refer to only a singular entity, but include the general class of which a specific example is used for illustration. The terminology herein is used to describe specific embodiments of the invention, but their usage does not limit the invention.

The following definitions are provided for specific terms that are used in the following written description.

As used herein a “suite of computer program products” refers to a group of program products that are compatible for exchanging data and interacting with each other.

As used herein, a “computer program product” refers to the expression of an organized set of instructions in the form of natural or programming language statements that is contained on a physical media of any nature (e.g., written, electronic, magnetic, optical or otherwise) and that may be used with a computer or other automated data processing system of any nature (but preferably based on digital technology). Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents. The term “software” and “computer program product” may be used interchangeably herein. Computer readable medium includes but not limited to: hard disks, floppy disks, compact disks, DVD's, flash memory, online internet web site, intranet web site; other types of optical, magnetic, or digital, volatile or non-volatile storage medium.

As used herein, “computer readable medium” includes cooperating or interconnected computer readable media, which exist exclusively on single computer system or are distributed among multiple interconnected computer systems that may be local or remote

As used herein, “a program” or “computer program” is generally a syntactic unit that conforms to the rules of a particular programming language and that is composed of declarations and statements or instructions, divisible into, “code” needed to solve or execute a certain function, task, or problem. A programming language is generally an artificial language for expressing programs.

As used herein, a “routine” refers to a section of a computer program comprising program language instructions for performing a particular task. For example, a data acquisition program according to the invention may further comprise an analysis routine. The term “routine” is used interchangeably herein with the term “procedure”, “function” and “subroutine”.

As used herein, “a computer system” of the invention generally comprises a central processing unit (CPU), which executes one or more programs embedded in a computer readable medium (i.e., a computer program product) to control the functions and/or properties of a microfluidic substrate. The systems according to the invention can include a stand-alone computer unit or several interconnected units. A functional unit is considered an entity of hardware or software, or both, capable of accomplishing a specified purpose. Hardware includes all or part of the physical components of the system, such as computers and peripheral devices. In one aspect, a CPU of the system executes a server program that receives and fulfills requests from client computers to execute instructions of computer program products according to the invention.

As used herein, “external system hardware” refers to hardware such as comprised in a microprocessor or controller which is in communication with a macroscale device (e.g., a stage, a pump, a micropositioner, and the like) which interfaces, either directly, or indirectly, with a microfluidic substrate. For example, “external system hardware” may include a microprocessor associated with a drive which communicates with a scanning table such as the stage of a microscope.

As used herein, the term “database” is used to include repositories for raw or compiled data, even if various informational facets can be found within data fields. A database is typically organized so its contents can be accessed, managed, mined, and updated (e.g., the database is dynamic). Preferably, the system according to the invention comprises a relational database comprising objects corresponding to functions and/or properties of one or more of: the microfluidic substrate, a sensor in fluid communication with the microfluidic substrate, functions and/or properties of one or more macroscale devices in communication with the microfluidic substrate, and/or objects corresponding to data from external databases, e.g., such as Medline, GenBank, AGTSDR: Agency for Toxic Substances and Disease Registry database, ChemFinder.com database, Alliance For Cellular Signaling (AFCS) database, Swiss-Prot, Kyoto Encyclopedia of Genes and Genomes (KEGG), Enzyme and co-factor database, other relational databases (e.g., such as bioinformatics databases) and the like. The relational database may be stored on a client computer (e.g., in the same room as the microfluidic substrate) or on a server computer which the client computer can access.

As used herein, a “microchannel” refers to a groove in a substrate comprising two walls, a base, at least one inlet and at least one outlet. In one aspect, a microchannel also has a roof. The term “micro” does not imply a lower limit on size, and the term “microchannel” is generally used interchangeably with “channel”. Preferably, a microchannel ranges in size from about 0.1 μm to about 500 μm, more preferably a microchannel ranges from 0.01 to about 150 μm.

As used herein, a “microfluidic substrate” refers to a substrate that comprises at least one microchannel. Generally, the specific size and geometry of the substrate is not limiting, however, preferably, a microfluidic substrate is of microscale dimensions (e.g., less than about 1 mm in at least one dimension, and preferably less than about 1 mm in all three dimensions). A substrate can be substantially planar, but may be of any shape—square, rectangular (i.e., in the form of a chip), circular, oblong, polygonal, etc. In some aspects, at least a portion of the substrate is not planar but has raised surface features, e.g., such as elevations (for example, for impaling a cell), at least two microchannels whose longitudinal axes are in different planes, interconnecting element(s) for interfacing the microfluidic substrate with a macroscale component, etc.

As used herein, a “chamber” refers to an area formed by walls (which may or may not have openings) surrounding a base. A chamber may be “open volume” (e.g., uncovered) or “closed volume” (e.g., covered by a coverslip, for example). A “sensor chamber” is one which receives one or more sensors and comprises outlets in one or more walls from at least two microchannels. However, a sensor chamber according to the invention generally can receive one or more nanoscopic or microscopic objects, without limitation as to their purpose. A sensor chamber can comprise multiple walls in different, not necessarily parallel planes, or can comprise a single wall which is generally cylindrical (e.g., when the chamber is “disc-shaped”). It is not intended that the geometry of the sensor chamber be a limiting aspect of the invention. One or more of the wall(s) and/or base can be optically transmissive. Generally, a sensor chamber ranges in size but is at least about 1 μm. In one aspect, the dimensions of the chamber are at least large enough to receive at least a single cell, such as a mammalian cell. The sensor chamber also can be a separate entity from the substrate comprising the microchannels. For example, in one aspect, the sensor chamber is a petrie dish and the microchannels extend to a surface of the substrate opening into the petrie dish so as to enable fluid communication between the microchannels and the petrie dish.

As used herein, a “sensor” refers to a device comprising one or more molecules capable of producing a measurable response upon interacting with a condition in an aqueous environment to which the molecule is exposed (e.g., such as the presence of a compound which binds to the one or more molecules). In one aspect, the molecule(s) are immobilized on a substrate, while in another aspect, the molecule(s) are part of a cell (e.g., the sensor is a “cell-based biosensor”).

As used herein, “a nanoscopic or microscopic object” is an object whose dimensions are in the nm to mm range.

As used herein, the term, “a cell-based biosensor” refers to an intact cell or a part of an intact cell (e.g., such as a membrane patch) which is capable of providing a detectable physiological response upon sensing a condition in an aqueous environment in which the cell (or part thereof) is placed. In one aspect, a cell-based biosensor is a whole cell or part of a cell membrane in electrical communication with an electrically conductive element, such as a patch clamp electrode or an electrolyte solution.

As used herein, the term “receptor” refers to a macromolecule capable of specifically interacting with a ligand molecule. Receptors may be associated with lipid bilayer membranes, such as cellular, golgi, or nuclear membranes, or may be present as free or associated molecules in a cell's cytoplasm or may be immobilized on a substrate. A cell-based biosensor comprising a receptor can comprise a receptor normally expressed by the cell or can comprise a receptor which is non-native or recombinantly expressed (e.g., such as in transfected cells or oocytes).

As used herein, “periodically resensitized” or “periodically responsive” refers to an ion-channel which is maintained in a closed (i.e., ligand responsive) position when it is scanned across microchannel outlets providing samples suspected or known to comprise a ligand. For example, in one aspect, an receptor or ion-channel is periodically resensitized by scanning it across a plurality of interdigitating microchannels providing alternating streams of sample and buffer. The rate at which the receptor/ion microchannel is scanned across the interdigitating microchannels is used to maintain the receptor/ion-channel in a ligand-responsive state when it is exposed to a fluid stream comprising sample. Additionally, or alternatively, the receptor/ion channel can be maintained in a periodically resensitized state by providing pulses of buffer, e.g., using one or more superfusion capillaries, to the ion channel, or by providing rapid exchange of solutions in a sensor chamber comprising the ion channel.

As used herein, the term “substantially separate fluid streams” refers to collimated streams with laminar flow.

As used herein, the term “in communication with” refers to the ability of a system or component of a system to receive input data from another system or component of a system and to provide an output response in response to the input data. “Output” may be in the form of data, or may be in the form of an action taken by the system or component of the system. For example, a processor “in communication with a scanning mechanism” sends program instructions in the form of signals to the scanning mechanism to control various scanning parameters as described above. A “detector in communication with a sensor chamber” refers to a detector in sufficient optical proximity to the sensor chamber to receive optical signals (e.g., light) from the sensor chamber. A “light source in optical communication with a sensor” refers to a light source in sufficient proximity to the sesnor to create a light path from the chamber to a system detector so that optical properties of the sensor can be detected by the detector. The term “in communication with” is used interchangeably with “operably linked” when the communication results in an action in response to input.

As used herein, “a measurable response” refers to a response that differs significantly from background as determined using controls appropriate for a given technique.

As used herein, an outlet “intersecting with” a chamber or microchamber refers to an outlet that opens or feeds into a wall or base or top of the chamber or microchamber or into a fluid volume contained by the chamber or microchamber.

As used herein, “superfuse” refers to washing the external surface of an object or sensor (e.g., such as a cell).

As used herein, “a function of a microfluidic substrate” refers to any operation, work, or step, performed done by a microfluidic substrate, or part thereof, either directly or when operably linked to another device. For example, a “function of a microfluidic substrate” may include, but is not limited to: fluid movement; separation; concentration; mixing; heating; focusing; and detection. A “function of a microfluidic substrate” may also include scanning a sensor relative to one or more channels in the substrate.

As used herein, “scanning of a sensor relative to one or more channels in a microfluidic substrate” refers to exposure of the sensor to a plurality of fluid streams from at least one channel in the substrate. This may be achieved by moving a sensor past one or more channel outlets in a stationary substrate providing such streams or by moving the substrate relative to a stationary sensor so that it is exposed to streams from one or more channel outlets of the substrate. Scanning may also be achieved by moving both the substrate and the sensor. Exposure to a plurality of fluid streams from a single channel may be achieved by providing different fluid streams (e.g., comprising different agents, or different doses of the same agent, or alternating buffer flow and flow of fluid stream containing an agent, or some combination thereof) from the single channel and/or by intermittently stopping the flow of fluid from an outlet of the channel in proximity to the sensor. In an embodiment where the sensor is stationary, scanning can be done by varying pressure at one or more channels. Combinations of the above scanning mechanisms may be used during a scanning process and variations of such combinations are obvious and encompassed within the scope of the invention.

As used herein, a “property of a microfluidic substrate” refers to a characteristic or feature of a microfluidic substrate or fluids in communication with the substrate (e.g., such as in a channel or reservoir or chamber in the substrate). A property of a microfluidic substrate may also refer to a characteristic or feature of an interface between the substrate and a macroscale device. Substrate properties include, but are not limited to: number of microchannels in the substrate; microchannel geometry; distance between microchannel outlets; number and/or location of reservoirs, chambers and/or sensors in contact with the substrate.

As used herein, “a macroscale device” refers to a device that is larger in at least one dimension compared with a microfluidic substrate with which it interfaces. As used herein, an “interface” between a microfluidic substrate and macroscale device is a contact point between a surface of the macroscale device and a surface of the microfluidic substrate and/or the contact between a surface of the macroscale device and a fluid in communication with the substrate.

As used herein, a data acquisition system “operably linked” to a microfluidic substrate refers to a system comprising a long term and/or short term memory (e.g., such as a cache) which provides instructions to an actuator in communication with the substrate which causes the substrate to execute one or more substrate functions and/or to change substrate properties (e.g., such as temperature) in response to receipt of the instructions. In one aspect, the data acquisition system is dynamic, causing the actuator to modify substrate functions and/or properties in real-time as data is received. In other aspects, substrate functions and/or properties are altered at pre-programmed or selected time intervals. An actuator may include, but is not limited to: a motor (e.g., piezo electric motor, molecular motor, etc), a switch (e.g., a microswitch), pump, resonator, micropositioner, valve, septum, nano-electromechanical device, a voltage or current source, a light source, a radiofrequency source, heat source, and the like. An actuator may be in direct communication with the microfluidic substrate. For example, an acuator may be an integral part of the substrate or removable component of the substrate. Alternatively, an actuator may be in indirect communication with the substrate. For example, the actuator may cause a macroscale device in communication with the substrate to perform an operation on the substrate (e.g., such as fluid and/or agent delivery, varying pressure, and the like).

Similarly, as used herein, a computer program product which is “operably linked” to a microfluidic substrate is one which provides instructions (e.g., through a processor providing signals to the actuator) which are executed by a actuator in communication with the substrate, which causes the substrate to execute one or more substrate functions and/or to change substrate properties in response to receipt of the instructions.

As used herein, “a parameter” of a property or function is a characteristic or attribute of a property or function that may be represented in the form of text or a numerical value.

Automated Microfluidic Workstation

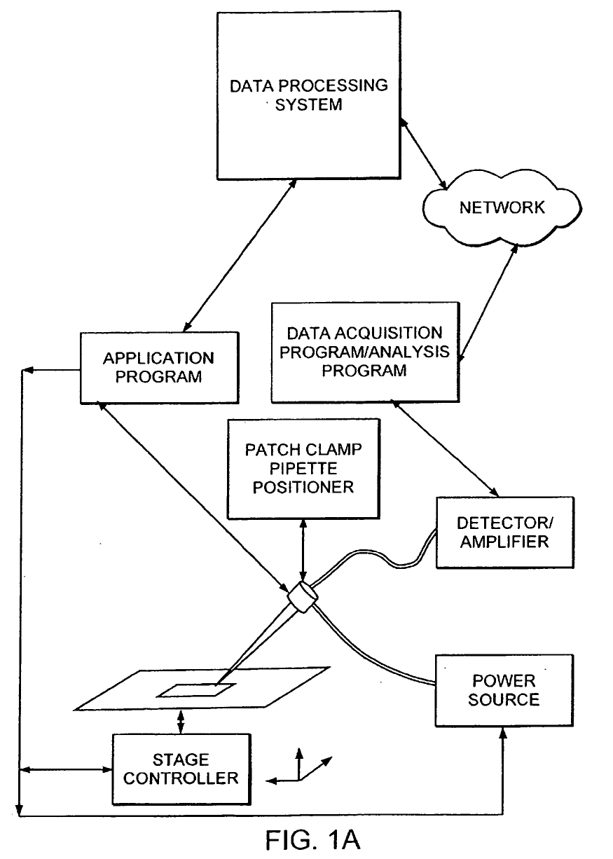

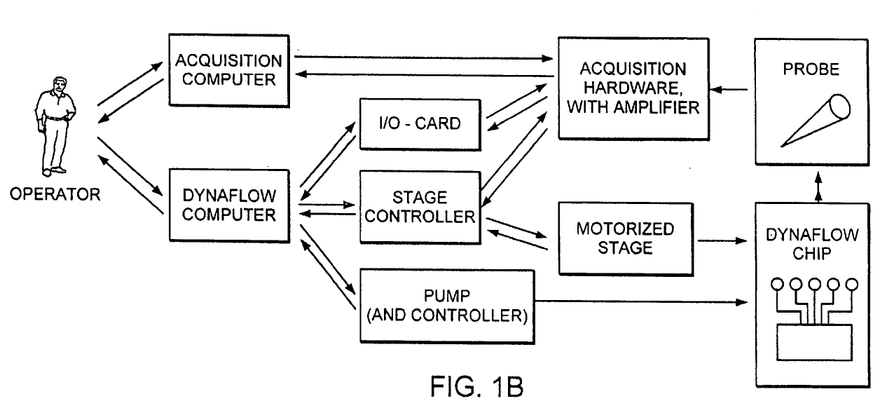

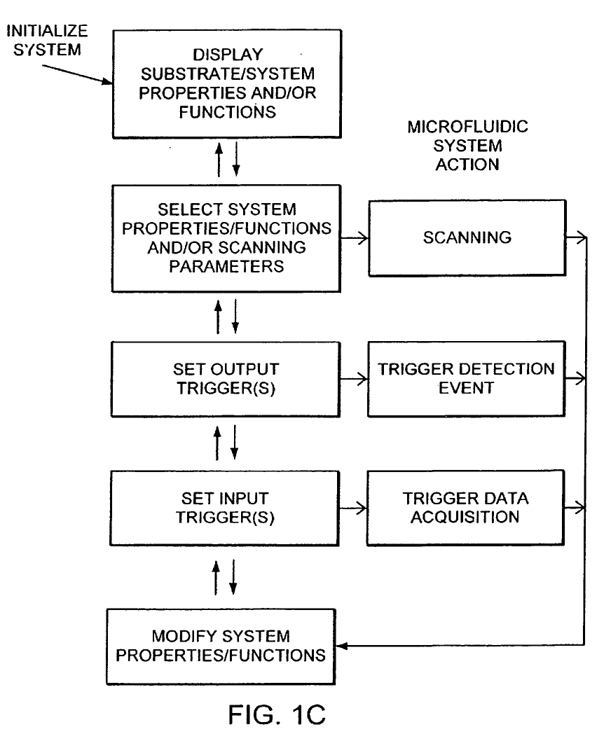

In one aspect, as shown in FIG. 1, a microfluidic workstation comprises a microfluidic substrate and a suite of computer program products for controlling and detecting processes occurring on a microfluidic substrate.

Preferably, the suite comprises a application program product that enables a user to specify one or more properties of a microfluidic substrate (e.g., microchannel number, inter-channel distance, etc.) and to control one or more functions at the substrate. Preferably, at least one substrate function includes scanning of the substrate relative to one or more sensors (e.g., by moving the substrate, by moving the one or more sensors, or by moving both the substrate and one or more sensors, or by varying pressure in one or more channels). Movement may be in an x-, y-, and/or z-direction. Alternatively, or additionally, movement may comprise rotating and/or tilting the substrate and/or sensor.

The microfluidic application program is constructed to receive data regarding one or more substrate properties (e.g., such as microchannel location data); to store the data; to access the data in response to a user signal or signal from the workstation; and to send instructions to external system hardware to perform one or more substrate functions (e.g., such as scanning). In one aspect, the microfluidic application program also receives data regarding parameters of desired substrate functions (e.g., number of microchannels to scan, time to complete scanning and the like). For example, the user may input the data into one or more fields on a display screen and/or may select options presented on a display screen (e.g., by clicking on a dropdown menu or by checking a box or selecting a button) to provide data to the application program. The application program then sends instructions to the external system hardware to execute actions corresponding to these parameters. Preferably, the application program communicates with a data processing system comprising a memory (e.g., such as a PC).

In another aspect, the workstation further comprises a data acquisition program for storing data received from at least the application program, in a memory unit. More preferably, the data acquisition system also receives data from detection software which has received data from the one or more sensors.

In one preferred embodiment, the one or more sensors comprise one or more cells and the acquisition program comprises patch clamp software such as Clampex (available from Axon Instruments, Union City, Calif.) or Pulse (available from HEKA Electronik, Lambrecht/Pfalz, Germany). The acquisition program may additionally provide analysis functions, e.g., such as Clampfit (Axon Instruments) or PULSEFIT, PULSET TOOLS, TIDA, and the like (HEKA Electronik).

Microfluidic Substrates

Microfluidic systems provide ways to manipulate minute volumes of liquid and to miniaturize assays involving the separation and detection of molecules. A microfluidic chip typically comprises a plurality of microchannels through which picoliter-to-nanoliter volumes of solvent, sample, and reagents solutions progress through narrow tunnels to be mixed, separated, and/or analyzed. Miniaturization increases performance and throughput, offering the potential for high throughput parallel processing. Because microfluidic devices can be designed to conform to microplate design standards, laboratories can work with robotic equipment used for dispensing samples and reagents into microwells of microplates can be adapted for use with these devices. Chips can be stacked to provide multi-dimensional microchannel networks. Microfluidic devices have applications in the processing and/or analysis of chemical reagents, nucleic acids, proteins, and cells.

In one aspect, a microfluidic substrate comprises a plurality of microchannels fabricated thereon whose outlets intersect with, or feed into, a sensor chamber comprising one or more sensors. In a preferred aspect, the sensor chamber comprises a cell-based biosensor in electrical communication with an electrode and the detector detects changes in electrical properties of the cell-based biosensor.

In a preferred aspect, the system comprises a substrate that delivers solutions to one or more sensors at least partially contained within a sensor chamber. The substrate can be configured as a two-dimensional (2D) or three-dimensional (3D) structure, as described further below. The substrate, whether 2D or 3D, generally comprises a plurality of microchannels whose outlets intersect with a sensor chamber that receives the one or more sensors. The base of the sensor chamber can be optically transmissive to enable collection of optical data from the one or more sensors placed in the sensor chamber. When the top of the sensor chamber is covered, e.g., by a coverslip or overlying substrate, the top of the chamber is preferably optically transmissive.

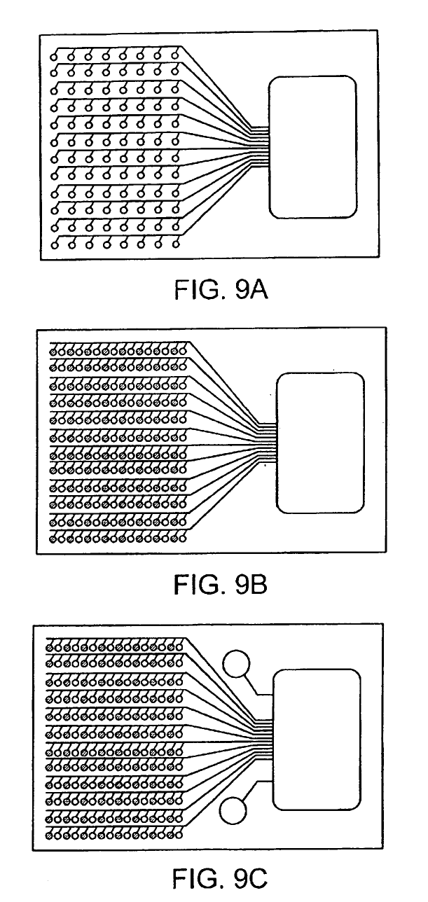

Each microchannel comprises at least one inlet (e.g., for receiving a sample or a buffer). Preferably, the inlets receive solution from reservoirs (e.g., shown as circles in FIGS. 9A and B) that conform in geometry and placement on the substrate to the geometry and placement of wells in an industry-standard microtiter plate.

In certain aspects, it is desirable to provide a substrate comprising an array of electrodes, e.g., to perform arrayed patch clamping. Microfabrication techniques are ideal for producing very large arrays of electrode devices. For example, electrode devices comprising nanotips can be manufactured by direct processing of a conducting solid-state material. Suitable solid-state materials include, but are not limited to, carbon materials, indium tin oxide, iridium oxide, nickel, platinum, silver, or gold, other metals and metal alloys, solid conducting polymers or metallized carbon fibers, in addition to other solid state materials with suitable electrical and mechanical properties. In one aspect, the substrate comprises an electrically conductive carbon material, such as basal plane carbon, pyrolytic graphite (BPG), or glassy carbon.

In one aspect, a cover layer of an optically transmissive material, such as glass, can be bonded to a substrate, using methods routine in the art, preferably leaving openings over the reservoirs and over the sensor chamber when interfaced with a traditional micropipette-based patch clamp detection system. Preferably, the base of the sensor chamber also is optically transmissive, to facilitate the collection of optical data from the sensor.

Microchannel Geometry

The body structure of the microfluidic devices described herein can take a variety of shapes and/or conformations, provided the body structure includes at least one microfluidic microchannel element disposed within it. For example, in some cases the body structure has a tubular conformation, e.g., as in a capillary structure. Alternatively, body structures may incorporate non-uniform shapes and/or conformations, depending upon the application for which the device is to be used. In preferred aspects, the body structure of the microfluidic devices incorporates a substantially planar or “chip” structure. In another aspect, discussed further below, the body structure comprises a “spokes-wheel” configuration and/or is substantially circular.

In one preferred aspect, the microfluidic substrate comprises a plurality of microchannels corresponding in number to the number of wells in an industry-standard microtiter plate to which the microchannels will be interfaced, e.g., 96 microchannels. When the system is used to provide alternating streams of sample and buffer to a sensor, at least 96 sample and 96 buffer microchannels (for a total of at least 192 microchannels) are provided. Wells of a microtiter plate, or of another suitable container, are coupled to reservoirs which feed sample or buffer to microchannels, e.g., for the system described previously, the substrate comprises 192 reservoirs, each reservoir connecting to a different microchannel. Additional reservoirs can be provided for cell storage and delivery, e.g., to provide cells for patch clamp recordings.

In one embodiment, microchannels are substantially parallel, having widths of about 100 μm and thicknesses of about 50 μm. The exact thickness of microchannels may be varied over a wide range, but preferably is comparable to, or greater than, the diameter of the sensor, e.g., the diameter of a patched cell. For example, inter-microchannel spacings of about 10 μm may be provided. However, as discussed further below, microchannels may additionally be non-parallel (e.g., radiating outward from a central sensor chamber).

Pressure can be applied simultaneously to all microchannels such that a steady state flow of solutions is made to flow through all microchannels at the same rate into the open volume that houses the sensor. In this way, steady state concentrations of different solutions containing ligands or pure buffer can be established at the immediate outlet of each of the microchannels. The width of each microchannel may be adjusted to achieve the desired flow rate in each microchannel.

Although the fluid streams exiting from the parallel microchannels enter an open volume sensor chamber in the embodiment discussed above, it may be more convenient and desirable to provide a set of parallel drain microchannels opposite the set of sample and buffer microchannels. A groove having an appropriate width (e.g., about 50 μm) can be placed in between, and orthogonal to, the two sets of microchannels (i.e., the delivery and drain microchannels) to accommodate scanning of a sensor in the sensor chamber. To establish an appropriate flow profile, a negative pressure may be applied to all the drain microchannels while simultaneously applying a positive pressure to the delivery microchannels. This induces fluid exiting the delivery microchannels to enter the set of drain microchannels.

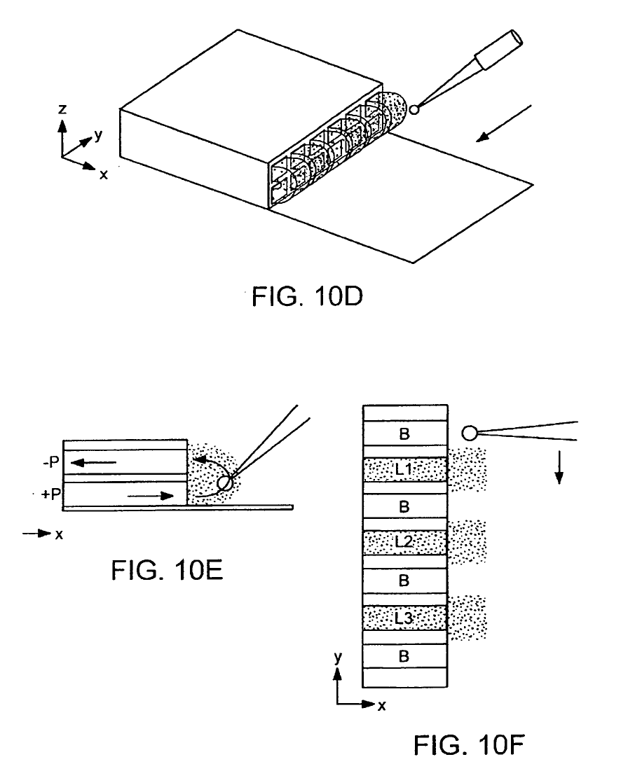

FIGS. 10A-C shows a three-dimensional microfluidic system. The main difference between this 3D structure and the planar structure shown in FIGS. 9A-C is the displacement along the z axis of fluid flowing between the outlet of the parallel array microchannels (e.g., interdigitated sample and buffer microchannels) and the inlet of the waste microchannels. In this embodiment, a positive pressure is applied to all sample and buffer microchannels while a negative pressure is simultaneously applied to all waste microchannels. Consequently, a steady state flow is established between the outlets of the sample/buffer microchannels and the inlets of the waste microchannels. In this configuration, a sensor, such as a patch-clamped cell, is scanned across the z-direction flow of fluid, preferably close to the outlet of the sample/buffer microchannels.

Although the fabrication of this 3D structure is more complex than the planar structure, the presence of z-direction flow in many cases will provide better flow profiles (e.g., sharper concentration gradients) across which to scan a sensor, such as a patch-clamped cell. The length over which z-direction flow is established should be significantly greater than the diameter/length of a sensor used. For example, the length of z-direction flow of a cell-based biosensor, such as a patch-clamp cell, should preferably range from about 10 μm to hundreds of μm.

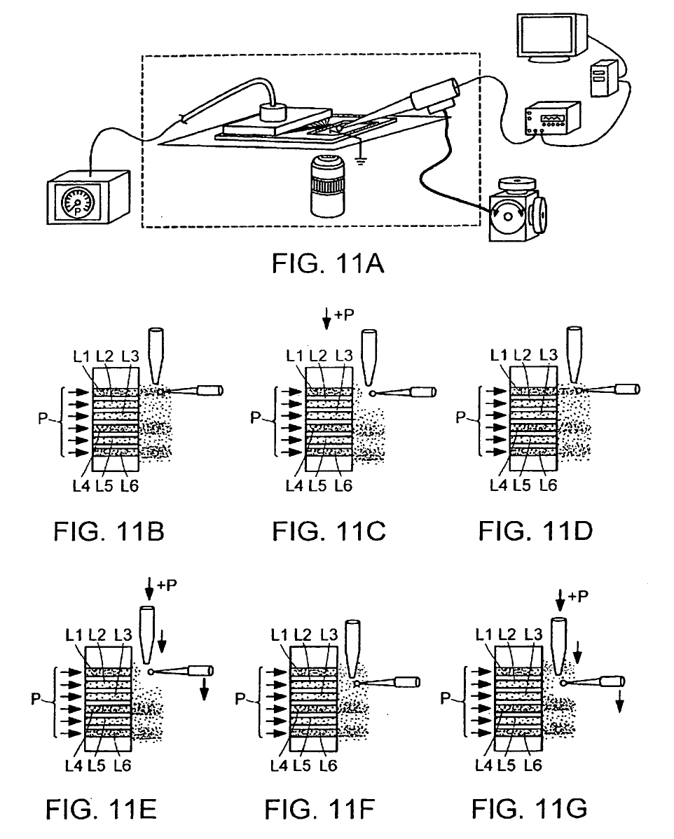

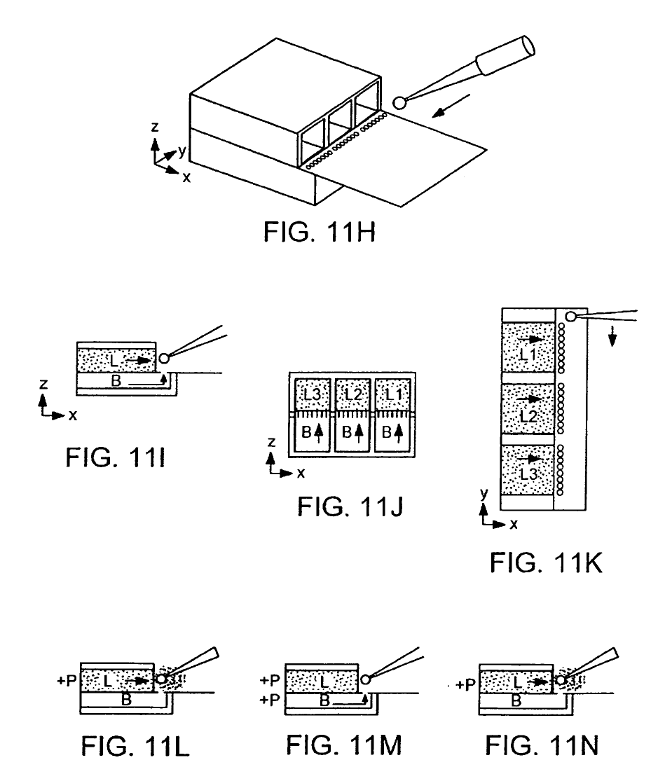

Another strategy for providing alternating sample streams and buffer streams, in addition to scanning, is shown in FIGS. 11A-N. In this embodiment, rather than providing interdigitating outlets which feed sample and buffer, respectively, into the sensor chamber, all outlet streams are sample streams. Buffer superfusion is carried out through one or more capillaries placed in proximity to one or more sensors. In FIG. 11A, the sensor shown is a patch-clamped cell positioned in proximity to an outlet using a patch clamp pipette. A capillary is placed adjacent to the patch clamp pipette and can be used for superfusion, e.g., to resensitize a desensitized cell. By this means, a cell-based biosensor comprising an ion microchannel can be maintained in a periodically responsive state, i.e., toggled between a ligand non-responsive state (e.g. bound to an agonist when exposed to drugs) and a ligand responsive state (e.g. ligand responsive after superfusion by buffer). Programmed delivery of buffer through this co-axial or side-capillary arrangement can be pre-set or based on the feedback signal from the sensor (e.g., after signal detection, buffer superfusion can be triggered in response to instructions from the system processor to wash off all bound ligands), providing pulsed delivery of buffer to the sensor. In one aspect, the longitudinal axis of the capillary is at a 90° angle with respect to the longitudinal axis of a patch clamp micropipette, while in another aspect, the longitudinal axis, is less than 90°.

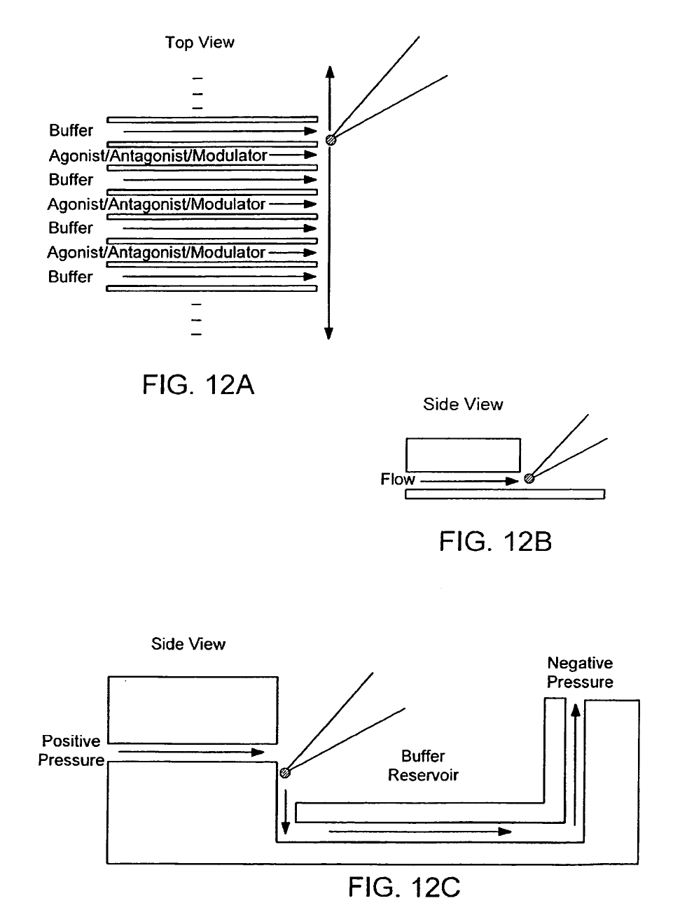

Microchannel outlets themselves also may be arranged in a 3D array (e.g., as shown in FIGS. 12A-F). A 3D arrangement of outlets can increase throughput (e.g., increasing the number of samples that can be screened) and therefore increase the amount of biological information that the sensor can evaluate. In one aspect, the microfluidic system is used to obtain pharmacological information relating to cellular targets, such as ion channels.

The microchannel geometry of the microfluidic device is not limiting. In one aspect, a plurality of microchannels converge or feed into the sensor chamber, while in another aspect, a plurality of microchannels converge into a single microchannel which itself converges into the sensor chamber. The plurality of microchannels can comprise interdigitating microchannels for sample and buffer delivery respectively.

Fluid Flow

Fluid flow in the microfluidic substrate can be controlled using a variety of methods.

Scheme 1: Using Septums to Address Individual Microchannels

In this scheme, the reservoirs that connect to each of the microchannels are sealed by a septum, for example, using polydimethyl siloxane (PDMS) for sealing or another suitable material as is known in the art. Because the septum forms an airtight seal, application of a positive pressure (e.g., with air or nitrogen) via a needle or a tube inserted through the septum will cause fluid to flow down the microchannel onto one or more sensors in a sensor chamber (e.g., to the center of a spokes-wheel where radial microchannels converge). Application of a negative pressure with a small suction through the needle or tubing inserted through the septum will cause fluid to be withdrawn in the opposite direction (e.g., from the chamber at the center of the spokes-wheel to the reservoir feeding into the microchannel).

An array of such needle-septum arrangements allows each reservoir to be individually addressed, and therefore, each microchannel. The use of this scheme permits the simultaneous and sequential pumping and valving of the fluids contained within each of the microchannels. By exercising precise control over positive and negative pressure applied to each of the microchannels, controlled fluid flow and compound delivery onto the one or more sensors can be achieved. For designs that do not require individual addressing of the microchannels (e.g., design 1—the rapid transport of patched cells across different streams of fluids), a single or a few septa with a single or a few pressure control devices will suffice.

Scheme 2: Controlling Fluidic Resistance by Varying Microchannel Dimensions

Although the above design using individual septa offers great flexibility and control, for certain applications in which the sequence of compound delivery and fluid flow is predetermined, a simpler design offers simplicity and ease of implementation. In this scheme, equal positive pressure is applied to all reservoirs, for example, by using pressurized air applied homogeneously to all reservoirs via a single septum, or through the use of gravity flow caused by the difference in height between inlet and outlet reservoirs. The rapid sequential delivery of compounds from each microchannel onto one or more sensors is accomplished by varying the fluidic resistance of each microchannel, which is easily achieved by varying the width and length of the each microchannel.

Fluidic resistance increases linearly with length and to the fourth power of the diameter for a circular capillary. By gradually and systematically varying the dimension of each microchannel, the time delay among the microchannels in their delivery of compounds onto one or more sensors in a sensor chamber can be controlled. This scheme is especially pertinent to high-throughput drug screening applications in which a large number of compounds are to be delivered sequentially and rapidly onto patched cell/cells with pre-determined time delays.

Scheme 3: Control of Fluid Flow With External Valves

In this configuration, compounds from each of the wells of an array well plate are introduced through external tubings or capillaries which are connected to corresponding microchannels. External valves attached to these external tubings or capillaries can be used to control fluid flow. A number of suitable external valves exist, including ones actuated manually, mechanically, electronically, pneumatically, magnetically, fluidically, or by chemical means (e.g., hydrogels).

Scheme 4: Control of Fluid Flow With Internal Valves

Rather than controlling fluid flow with external valves, there are also a number of chip-based valves that can be used. These chip-based valves can be based on some of the same principles used for the external valves, or can be completely different, such as ball valves, bubble valves, electrokinetic valves, optically controlled valves, diaphragm valves, and one-shot valves. The advantage of using chip-based valves is that they are inherently suited for integration with microfluidic systems. Of particular relevance are passive one-way valves, which are preferred for implementing some of the designs mentioned in above (e.g., such as the branched microchannel format).

Electrical potential differences may also be used to move fluids in the microchannels of the substrate. For example, electrophorosmosis or dielectrophoresis can be used. See, e.g., U.S. Pat. No. 5,632,876; U.S. Pat. No. 5,992,820; U.S. Pat. No. 5,800,690, and U.S. Pat. No. 6,001,231.

Cell-Based Biosensors

In one aspect, the microfluidic system is used in conjunction with a cell-based biosensor to monitor a variety of cellular responses. The biosensor can comprise a whole cell or a portion thereof (e.g., a cell membrane patch) which is positioned in a sensor chamber using a micropositioner (which may be stationary or movable) such as a pipette, capillary, column, or optical tweezer, or by controlling flow or surface tension, thereby exposing the cell-based biosensor to solution in the chamber. The biosensor can be scanned across the various microchannels of the substrate by moving the substrate, i.e., changing the position of the microchannels relative to the biosensor, or by moving the cell (e.g., by scanning the micropositioner or by changing flow and/or surface tension).

In one aspect, the cell-based biosensor comprises an ion microchannel and the system is used to monitor ion microchannel activity. In another aspect, the cell-based biosensor comprises a receptor, preferably, a receptor involved in a signal transduction pathway. Biosensors expressing recombinant receptors also can be designed to be sensitive to drugs which may inhibit or modulate the development of a disease.

In one aspect, the substrate provides one or more cell treatment chambers for performing one or more of: electroporation, electroinjection, and/or electrofusion. Chemicals and/or molecules can be introduced into a cell within a chamber which is in electrical communication with a source of current. For example, one or more electrodes may be placed in proximity to the chamber, or the chamber can be configured to receive an electrolyte solution through which current can be transmitted, e.g., from an electrode/capillary array as described in WO 99/241 10, the entirety of which is incorporated by reference herein.

Suitable molecules which can be introduced into a cell in the cell treatment chamber include, but are not limited to: nucleic acids (including gene fragments, cDNAs, antisense molecules, ribozymes, and aptamers); antibodies; proteins; polypeptides; peptides; analogs; drugs; and modified forms thereof. In a preferred aspect, the system processor controls both the delivery of molecules to the one or more cell treatment chambers (e.g., via capillary arrays as described above) and incubation conditions (e.g., time, temperature, etc.). For example, a cell can be incubated for suitable periods of times until a desired biological activity is manifested, such as transcription of an mRNA; expression of a protein; inactivation of a gene, mRNA, and/or protein; chemical tagging of a nucleic acid or protein; modification or processing of a nucleic acid or protein; inactivation of a pathway or toxin; and/or expression of a phenotype (e.g., such as a change in morphology).

The treated cells can be used to deliver molecules of interest to the sensor in the sensor chamber, e.g., exposing the sensor to secreted molecules or molecules expressed on the surface of the cells. In this aspect, the system can be programmed to release a cell from a cell treatment chamber into a microchannel of the system intersecting with the sensor chamber, thereby exposing a sensor in the sensor chamber to the molecule of interest.

Alternatively, or additionally, when the system is used in conjunction with a cell-based biosensor, the cell treatment chamber can be used to prepare the biosensor itself. In one aspect, a cell is delivered from the treatment chamber to a microchannel whose outlet intersects with the sensor chamber. In one aspect, the scanning mechanism of the system is used to place a micropositioner in proximity to the outlet so that the micropositioner can position the cell within the sensor chamber. In another aspect, fluid flow or surface tension is used to position a cell in a suitable position. For example, the system can be used to deliver the cell to the opening of a pipette which is part of a patch clamp system.

In another aspect, a cell can be delivered to the sensor chamber to periodically replace a cell-based biosensor in the sensor chamber. In this aspect, the cell can be untreated, e.g., providing a substantially genetically and pharmacologically identical cell (i.e., within the range of normal biological variance) as the previous sensor cell. Alternatively, the replacement cell can be biochemically or genetically manipulated to be different from the previous sensor cell, to enable the system to monitor and correlate differences in biochemical and/or genetic characteristics of the cells with differences in sensor responses. The biochemical or genetic difference can be known or unknown.

The system can be programmed to deliver cells from the cell treatment chamber at selected time periods based on control experiments monitoring uptake of chemicals and molecules by cells. Alternatively, the system can monitor the phenotype of cells and deliver cells when a certain phenotype is expressed. For example, in one aspect, the cell treatment chamber is in communication with an optical sensor which provides information relating to optical properties of the cell to the system processor, and in response to optical parameters indicating expression of a particular phenotype, the system can trigger release of the cell from the cell treatment chamber. Optical parameters can include the uptake of a fluorescent reporter molecule or optical parameters identified in control experiments.

A cell can be positioned in the measurement chamber using a micropositioner (which may be stationary or movable) such as a pipette, capillary, column, optical tweezer, piezoelectric cantilever systems and/or can be dispensed into a measurement chamber using a dispenser such as an nQUAD aspirate dispenser. Other methods can used to position a cell such as, suction, the use of voltage pulses (electrophoresis, dielectrophoresis, electroendoosmosis), and the like.

In one aspect, pressure-driven flow is used to manipulate the movement of cells from microfluidic microchannels in the substrate to the measurement chamber. Routing of cells can be affected by blocking a branch of a microchannel in a substrate comprising a plurality of microchannels, using valves as are known in the art (and discussed further below), thereby moving the cells along with bulk solution flow into another, selected microchannel or into the measurement chamber.

Additionally, or alternatively, electroosmosis can be used to produce motion in a stream containing ions, e.g., such as buffer solution, by application of a voltage differential or charge gradient between two or more electrodes. Neutral (uncharged) cells can be carried by the stream. See, e.g., as described in U.S. Published application No. 2002/0049389.

Dielectrophoresis produce movement of dielectric objects, which have no net charge, but have regions that are positively or negatively charged in relation to each other. Alternating, non-homogeneous electric fields in the presence of cells cause the cells to become electrically polarized and thus to experience dielectrophoretic forces. Depending on the dielectric polarizability of the particles and the suspending medium, dielectric particles will move either toward the regions of high field strength or low field strength. The polarizability of living cells depends on the type of cell and this may provide a basis for cell separation, e.g., by differential dielectrophoretic forces. See, e.g., as described in U.S. Published application 2002/0058332.

The cell chamber itself can be configured to include one or more electrical elements for creating an electrical field to aid in positioning cell(s) in proximity to an appropriate electrode compartment, e.g., to create electroosmotic flow within the cell chamber or to polarize a cell to facilitate its movement towards an electrode compartment.

In another aspect, a cell holder (e.g., a micropositioner) can be used to position the cell in a sensor chamber in proximity to an electrode device comprising a nanotip. Preferably, a cell holder comprises an end with an opening whose diameter is about the diameter of the cell, or less than 500 μm, and more preferably, less than about 100 μm, or less than about 50 μm. In one aspect, the diameter of the opening is slightly smaller than the cell, i.e., about 10 μm, or more preferably 5 μm. Suitable cell holders include capillaries or micropipettes and, as discussed above, cell holders can be moveable in an x-, y-, or z-direction and can be used in conjunction with electrode devices to measure the electrical properties of cells in suspension. Cells can be transiently stably associated with cell holders by moving the cell to the holder (e.g., using fluid flow, pressure differentials, electric fields, and/or optical tweezers) and applying a gentle suction on the cell holder or a small electric voltage.

The ability to combine of on-chip electroporation with microfluidics and patch clamp (or other methods for monitoring cell responses) facilitates screening for molecules (e.g., ligands or drugs) which modulate the activity of intracellular targets. In one aspect, the system is used to deliver a cell-impermeant molecule into the interior of a cell by transiently electroporating the cell. In this way, the molecule can be introduced to intracellular receptors, intracellular proteins, transcriptional regulators, and other intracellular targets. The cell can be delivered to the sensor chamber and the response of the cell can be monitored (e.g., by patch clamp or by fluorescence, if the molecule is tagged with a fluorescent label). Alternatively, the sensor chamber can be modified to perform both treatment and response detection.

In a further aspect, the system can be modified to perform electroporation by scanning. For example, a cell can be repeatedly electroporated as it is being translated or scanned across a plurality of different fluid streams containing different compounds. In one aspect, pores are introduced into one or more cells as they come into contact with a sample stream, enabling compounds in the sample stream to be taken up by the cell.

By controlling pressure, the system can control the velocity (both amplitude and direction) of fluid streams. Velocity control also may be exercised by controlling the resistance of each microchannel without changing the pressure or by changing both resistance and pressure. Fluid shear also can be varied by using solutions of different viscosity (e.g., adding different amounts of a sugar such as sucrose to a fluid stream) in both the microchannels and sensor chamber. Thus, by varying a number of different parameters, the flow profile of different fluid streams can be precisely tuned.

Non-Cell Based Biosensors

In one aspect, the sensor comprises a sensing element, preferably, a molecule which is cellular target (e.g., an intracellular receptor, enzyme, signaling protein, an extra cellular protein, a membrane protein, a nucleic acid, a lipid molecule, etc.), which is immobilized on a substrate. The substrate can be the base of the sensor chamber itself, or can be a substrate placed on the base of the chamber, or can be a substrate which is stably positioned in the chamber (e.g., via a micropositioner) and which is moveable or stationary.

The sensor may consist of one or several layers that can include any combination of: a solid substrate; one or more attachment layers that bind to the substrate, and a sensing molecule for sensing compounds introduced into the sensor chamber from one or more microchannel outlets. Suitable sensors according to the invention, include, but are not limited to, immunosensors, affinity sensors and ligand binding sensors, each of which can respond to the presence of binding partners by generating a measurable response, such as a specific mass change, an electrochemical reaction, or the generation of an optical signal (e.g., fluorescence, or a change in the optical spectrum of the sensing molecule). Such sensors are described in U.S. Pat. No. 6,331,244, for example, the entirety of which is incorporated by reference herein.

In one aspect, the sensor comprises a microelectrode which is modified with a molecule which transports electrons. In response to a chemical reaction caused by contact with one or more compounds in an aqueous stream from one of the microchannels, the molecule will produce a change in an electrical property at the electrode surface. For example, the molecule can comprise an electron-transporting enzyme or a molecule which transduces signals by reduction or oxidation of molecules with which it interacts (see, e.g., as described in, Gregg, et al., 1991, J. Phys. Chem. 95: 5970-5975, 1991; Heller, 1990, Acc. Chem. Res. 23(5): 128-134;Chap, 1994, In Diagnostic Biosensor Polymers. ACS Symposium Series. 556; Usmani, A M, Akmal, N; eds. American Chemical Society; Washington, D.C.; pp. 47-70; U.S. Pat. No. 5,262,035). Enzymatic reactions also can be performed using field-effect-transistors (FETs) or ion-sensitive field effect transistors (ISFETs).

In another aspect, the sensor comprises a sensing molecule immobilized on a solid substrate such as a quartz chip in communication with an electronic component. The electronic component can be selected to measure changes in any of: voltage, current, light, sound, temperature, or mass, as a measure of interaction between the sensing element and one or more compounds delivered to the sensor chamber (see, as described in, Hall, 1988, Int. J. Biochem. 20(4): 357-62; U.S. Pat. No. 4,721,677; U.S. Pat. No. 4,680,268; U.S. Pat. No. 4,614,714; U.S. Pat. No. 6,879,11). For example, in one aspect, the sensor comprises an acoustic wave biosensor or a quartz crystal microbalance, on which a sensor element is bound. In this embodiment, the system detects changes in the resonant properties of the sensor upon binding of compounds in aqueous streams delivered from the microchannels to the sensor element.

In another aspect, the sensor comprises an optical biosensor. Optical biosensors can rely on detection principles such as surface plasmon resonance, total internal reflection fluorescence (TIRF), critical angle refractometry, Brewster Angle microscopy, optical waveguide lightmode spectroscopy (OWLS), surface charge measurements, and evanescent wave ellipsometry, and are known in the art (see, for example, U.S. Pat. No. 5,313,264; EP-A1-0 067 921; EP-A1-0 278 577; Kronick, et al., 1975, J. Immunol. Meth. 8: 235-240).

For example, for a sensor employing evanescent wave ellipsometry, the optical response related to the binding of a compound to a sensing molecule is measured as a change in the state of polarization of elliptically polarized light upon reflection. The state of polarization is related to the refractive index, thickness, and surface concentration of a bound sample at the sensing surface (e.g., the substrate comprising the sensing element). In TIRF, the intensity and wavelength of radiation emitted from either natively fluorescent or fluorescence-labeled sample molecules at a sensor is measured. Evanescent wave excitation scattered light techniques rely on measuring the intensity of radiation scattered at a sensor surface due to the interaction of light with sensing molecules (with or without bound compounds). Surface plasmon resonance (SPR) measures changes in the refractive index of a layer of sensor molecules close to a thin metal film substrate (see, e.g., Liedberg, et al., 1983, Sensors and Actuators 4: 299;GB 2 197 068). Each of these sensing schemes can be used to provide useful sensors according to the invention.

In yet another aspect, the sensor comprises a sensing molecule associated with a fluorescent semiconductor nanocrystal or a Quantum Dot™ particle. The Quantum Dot particle has a characteristic spectral emission which relates to its composition and particle size. Binding of a compound to the sensing element can be detected by monitoring the emission of the Quantum Dot particle (e.g., spectroscopically) (see, e.g., U.S. Pat. No. 6,306,610).

The sensor further can comprise a polymer-based biosensor whose physical properties change when a compound binds to a sensing element on the polymer. For example, binding can be manifested as a change in volume (such as swelling or shrinkage), a change in electric properties (such as a change in voltage or current or resonance) or in optical properties (such as modulation of transmission efficiency or a change in fluorescence intensity).

Application Program for Programmably Controlling Processes in a Microfluidic Substrate

As shown in FIGS. 1A and 1B, the workstation preferably comprises a data processing unit which can implement the instructions of an application program. The application program can direct movement of a sensor in a sensor chamber of a microfluidic substrate. Preferably, the application program controls scanning of a sensor across the multiple microchannel outlets of the microfluidic substrate. The application program communicates with electronic hardware that directly controls this movement. In one aspect, this electronic hardware is a microprocessor associated with a scanning table (such as a stage of a microscope on which the substrate is placed) and/or with a micropositioner (such as for controlling the positioning of a sensor, e.g., a cell) in proximity to a sensor chamber of the substrate. In one aspect, the micropositioner is a microelectrode or patch clamp pipette that dips into a liquid media contained in the sensor chamber.

The microfluidic system additionally comprises a data acquisition system. The data acquisition system comprises hardware and software which monitors various actions of the microfluidic substrate and which measures and records signals from one or more sensors in sensor chambers of the microfluidic substrate. In one particularly preferred aspect, the acquisition system obtains and processes data from patch clamp recordings of one or more patch clamped cells.

The hardware and software may be part of a single central system memory or a central processing unit that also contains the application software. Alternatively, the software and hardware may communicate through a local area network (LAN) or wide area network (WAN).

The application system can be run on PC-based workstations as are known in the art. In one aspect, the application is run on a 500 MHz workstation with 256 Mb of RAM memory. The microfluidic system workstation may additionally include suitable operating system software, such as a Windows® platform (e.g., such as Windows® 2000 and Windows® XP) In general, the computer program products used in the workstation are not computer-specific and as technology evolves, the system can implement and adapt other program products and storage platforms. Assay specific programs can be implemented using standard C++ programming language or other suitable language.

The one or more of the programs of the suite may in turn communicate with external hardware with input output controllers (I/O's), e.g., through a system bus. External hardware is operably linked to macroscale devices which interface with the microscale microfluidic systems.

Macroscale devices include, but are not limited to: stages or scanning tables for scanning the microfluidic substrate; micropositioners in proximity to the substrate (e.g., cell holders, pipets, nanoelectrodes or electrode arrays); fluid delivery systems (e.g., tubing manifolds and valve systems); sensors (optical, temperature, chemical, electrical, pressure sensors); detectors; pump heads; pumps; separation devices; concentration devices; electroporator devices; electrical connecting elements, and the like.

Suitable input-output controllers are generally any devices which can accept and process information from a user, whether a human or machine, local or remote. Such devices include, but are not limited to: modem cards, network interface cards, sound cards, graphical user interface controllers other types of controllers for any of a variety of known input devices. Output controllers of input-output controllers include graphical user interface controllers for display devices such a user interface to logically and/or physically organized data/signals from a controller into an array of picture elements or pixels (i.e., the display).

Interfaces between macroscale devices and microscale devices can be in the form of mechanical fasteners. For example, the microfluidic substrate, in one aspect, comprises a chip socket that fits into an industry-standard microplate holder such as are available for most microscope stages. The chip socket can be used to mount the microfluidic substrate onto the stage. In other aspect, an adhesive tape can be used such as disclosed in U.S. Serial No. 60/417,342, filed Oct. 9, 2002).

The microfluidic application program product according to the invention can be used to programmably expose a sensor to a fluid stream (e.g., comprising an analyte and/or detection molecule). To this end, the application program can be installed onto the medium of a memory comprised in the system. For example, the installation can be started by running a setup program (setup.exe) such as is generally sold to software makers as tools for creating install programs for their software. Older versions of application systems are preferably removed prior to installation, e.g., such as using an ADD PROGRAM DIALOGUE or REMOVE PROGRAM DIALOGUE button displayed when accessing the system memory as implemented by a standard install program of a Windows-based operating system. After the install program is completed, an icon is displayed on the display of a user interface in communication with the system memory (e.g., in the start menu under PROGRAMS).

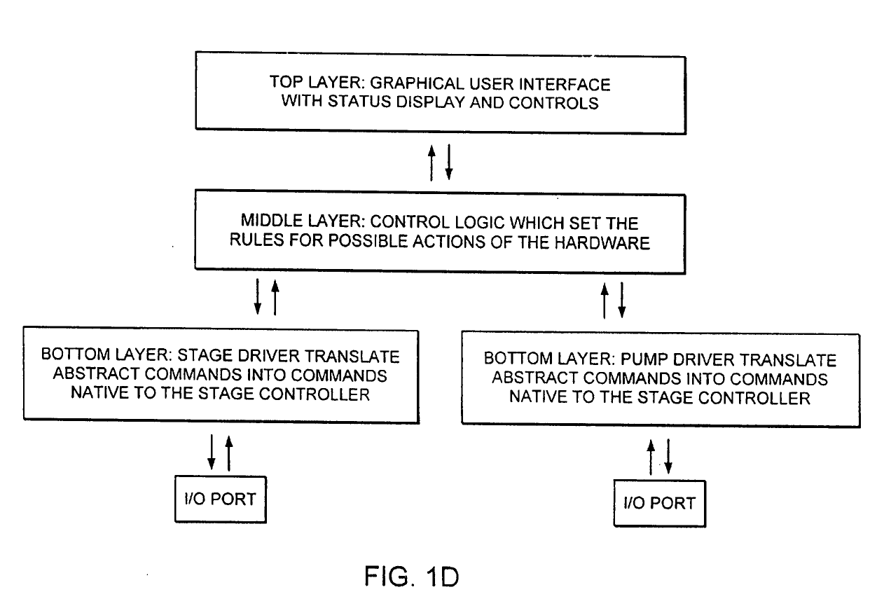

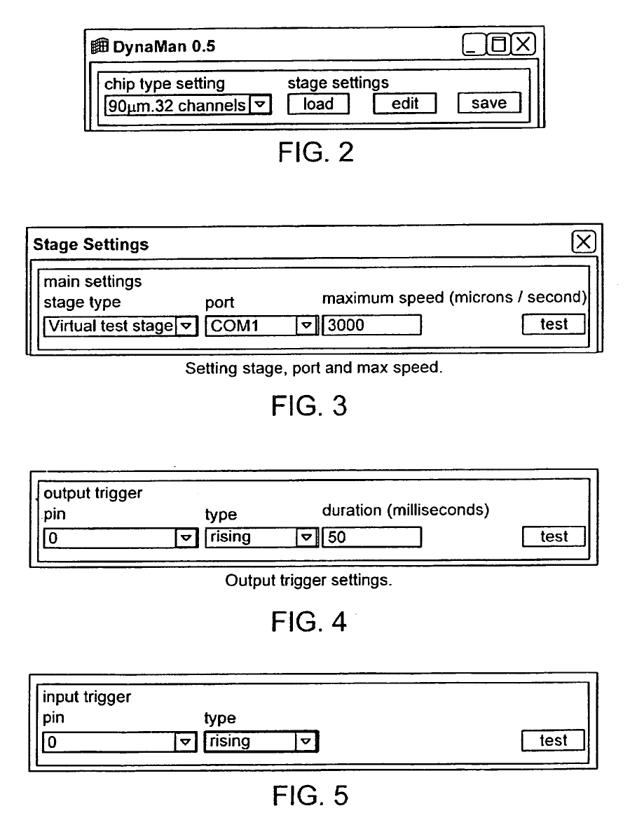

Preferably, the display interface displays a screen on which various substrate properties are displayed. For example, the screen may provide a selectable menu on which different types of substrate configurations are indicated. For example, as shown in FIG. 2, the user interface may display a drop down menu with the label CHIP TYPE. A substrate type should be selected which is the same type as the substrate being used. In one aspect, a CHIP TYPE identifier indicated in the drop down menu corresponds to the number of microchannels provided in the chip. However, other identifiers can be used which can be associated with particular types of chip geometry by the application program. Similarly, a plurality of identifiers can be used to identify a substrate type. For example, a set of identifiers can designate the number of microchannels leading to a sensor chamber in the chip for receiving a sensor, as well as identifying the geometry of the microchannels (e.g., parallel, fish bone, spokes-wheel, and the like). Preferably, this is done each time an application is selected. In one aspect, the substrate is coded with a bar code, a radiofrequency tag, or other identifier that is recognized by the microfluidic system (e.g., by a detector, receiver, and the like) and this triggers the user interface to display a display screen appropriate for the type of substrate. For example, upon recognizing a barcode, the system can display the numbers of channels, interchannel distances and other properties associated with the substrate identified by the barcode, the properties listed in a table comprised in a relational database which is part of the system.

A display screen is displayed which provides a dialog of configurable settings corresponding to the appropriate microfluidic substrate properties and/or possible system actions suitable for the substrate. Substrate properties include STAGE TYPE which provides a drop down list of stage types for selecting a driver suitable for the particular stage being used to scan the microfluidic substrate. Alternatively, or additionally, the system may include a MICROPOSITIONER TYPE menu providing a list of micropositioner types whose movement may be activated by the system.

In one preferred aspect, the application system may be programmed to provide instructions to a stage, such as one provided by Prior (e.g., such as a ProScan Stage) or by Martshauser (e.g., such as ECODRIVE or SCAN stages). The stage communicates with the application system through a controller, such as Corvus (available from Martshauser, Wetzlar-Steindorf, Germany) which in turn communicates with the application program of the system through an interface (including, but not limited to an RS-232 interface, an Ethernet- or TCP/IP interface, an scsi, usb or parallel port).

As shown in FIG. 3, in one aspect, a VIRTUAL TEST STAGE is provided as a selectable option on the list. The virtual test stage is suitable for educational or demonstration purposes and is coupled to a stage driver for providing a virtual system which fictions substantially like a real stage. The virtual stage preferably is associated with force feedback outputs (e.g., conveyed through joysticks and/or graphical displays, such as video images, and/or sound) to allow a user to experience manipulating a microfluidic system. The VIRTUAL TEST STAGE can be used to test the application system without a supported stage controller.

As shown in FIG. 3, an additional system property that can be selected is the stage port. In one aspect, a user selects a suitable serial port that can be used to control the movement of the scanner (e.g., stage). A port should be selected where the stage controller or driver is physically connected.

In another aspect, the selectable system property is the maximum speed at which a sensor moves relative to the substrate. For example, when using the substrate in a patch clamp application in which the sensor is a cell, a maximum speed at which the cell/sensor may move should be selected so that the cell/sensor does not move too quickly in the liquid on the microfluidic substrate (i.e., in the sensor chamber). Typically, the unit used is microns per seconds. A value of 3000 means that a maximum speed of 3 mm/second is used (see, e.g., FIG. 3).

If a value is entered which is greater than the highest speed the scanning mechanism (e.g., stage or micropositioner) is physically capable of, the application will create a default situation and all of the speeds of the stage/micropositioner will be available. Internally, the value is used to set the speed for one or more actions of the scanning mechanism, such as positioning a sensor to a preselected microchannel outlet (the MOVE command), scanning the sensor past a plurality of microchannel outlets in a sensor chamber (INPUT TRIGGER STEPPING scan mode), or scanning the sensor past a plurality of microchannels while including one or more pause intervals (ALTERNATING MICROCHANNEL DELAY scan mode). For a CONTINUOUS MOVEMENT scan, the maximum speed setting will limit the available list of options provided by a drop down menu labelled TIME/MICROCHANNEL which controls the amount of time a sensor pauses at any one microchannel. For example, if only slow speeds are allowed, only long time intervals in each channel will be selectable.

Preferably, the user interface also displays a TEST button so that when the stage, port, and maximum speed have been set, the settings may be evaluated by clicking on the TEST button. If a satisfactory result is obtained, the system can be activated to provide instructions to various components of the system to performed selected system actions.

In one aspect, output triggers are used to send signals to data acquisition hardware to synchronize the system action DETECTION (e.g., such as recording) with the system action MOVE or SCAN. For this embodiment, the scanning mechanism (e.g., stage controller or micropositioner) comprises an i/o port and a suitable stage driver recognized by the system. Output triggers can be used in all scan modes. FIG. 4 shows a user interface display for controlling output trigger settings. A pin appropriate for a particular connector being used is selected from a drop down menu. Another drop down menu provides a drop down list of selectable trigger types. The option RISING TRIGGER when implemented results in a trigger that is sent when a signal transits from a predefined low value to a predefined high value, while the option FALLING TRIGGER means that the trigger is sent when a signal transits from a predefined high value to a predefined low value. The DURATION setting determines how long (e.g., in msec) the trigger will be. By selecting an OUTPUT TRIGGER, a particular threshold signal can be associated with a scanning motion which can be used to trigger a detection event, such as a patch clamp recording.

To test the output trigger, the TEST button can be used. The result should be viewable as a positive signal or a trigger pulse displayed on an oscilloscope in communication with the system, or alternatively, can be displayed on a display receiving input from the data acquisition system or determined by using a multimeter as is known in the art.

Input trigger settings also may be used. For example, such triggers may be used to synchronize stage movement with data acquisition system instructions, for example, coordinating pauses by the scanning mechanism (e.g., stage movement) with external system triggers such as a measured condition within the system (e.g., arrival of a labelled analyte at a microchannel outlet, arrival of a cell at a microchannel outlet, fluid movement through one or more microchannels, an electroporation event, an electrophoresis event, a concentration or mixing event, and the like). Preferably, in this embodiment, the scanning mechanism has a suitable i/o port and stage driver recognized by the application system. As shown in FIG. 5, the input trigger display interface can include a pin type drop down menu and a type drop down menu (e.g., rising or falling). The input trigger may also be tested by selecting a TEST button. The scanning mechanism driver (e.g., such as the stage driver) will not activate until the system trigger is detected if the test works properly and a positive response to the application instructions associated with the selected input trigger settings is manifested as a positive response by the scanning mechanism (e.g., initiation of movement, change of movement parameters, or cessation of movement). If no trigger has been detected for given preselected time period (e.g., such as about 5 seconds) a timeout will occur and a negative response will be displayed.

When desired stage settings have been configured, these may be saved for future use by using a SAVE button displayed on the user interface.

Preferably, a microfluidic substrate according to the invention comprises reference points in proximity to the first and last microchannels. A user provides the coordinates to the application system which uses the reference points to calculate the locations of all of the remaining microchannels. Generally, reference points are set each time the application is started and each time a new substrate is inserted into the substrate socket and mounted on the stage. For example, the stage may be part of an optical system, such as an inverted microscope to provide a stage for the microfluidic substrate capable of moving an x-, y- or z-direction over the microscope's objective. A z-focusing drive may be used to initially image the substrate. Alternatively, or additionally, the stage may rotate (e.g., such as when the substrate is circular and/or comprises a spokes-wheel configuration). One or more joysticks can be used to provide for manual movement of the stage in the x-, y-, z-direction. Motion along all axes can be driven by stepper motors so that precise and accurate positioning may be achieved. A servo motor or other actuator systems may be used for precise position control.

A camera, preferably, a high digital resolution camera, acquires images from one or more locations on the substrate making the reference points on the substrate visible on the display of the user device. Reference points on the substrate can also be marked, e.g., with fluorescent markings, such that when the substrate is in the focal plane of a detector in the work station, the reference points can be displayed as images on the display of the user interface. In one aspect, the substrate is positioned on the stage of a scanning confocal laser microscope for facilitating this end. In another aspect, video images of a substrate are acquired and displayed on one or more display interfaces. Video images can be fed into the central processing unit via a frame-grabber card.

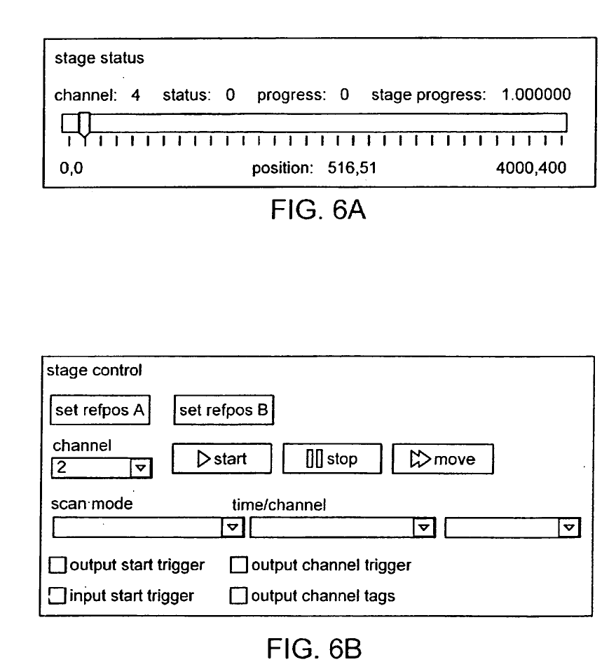

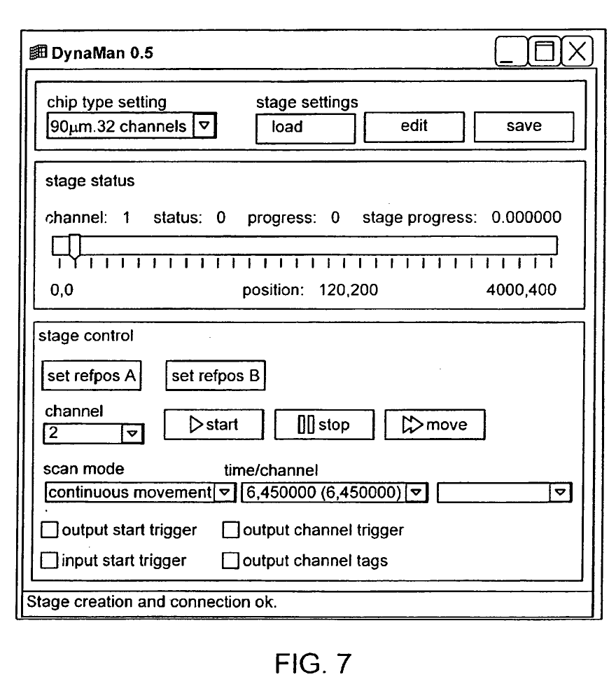

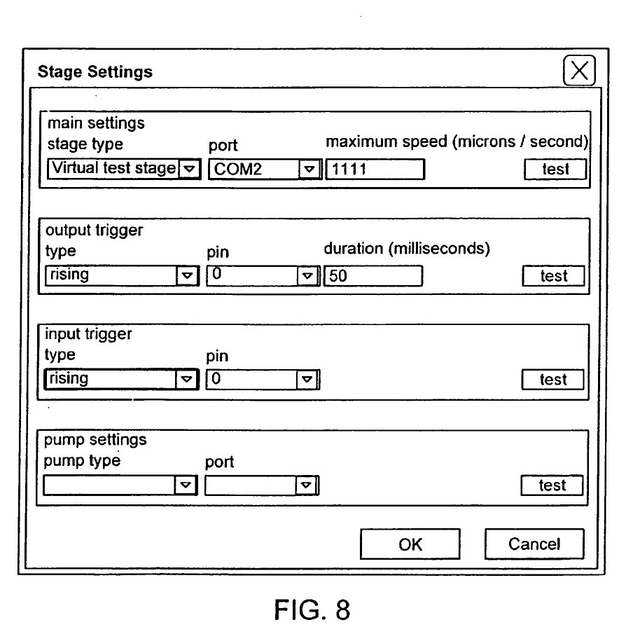

The stage is moved so that the micropositioner or probe is visible and in focus directly above both reference points, one at a time. Reference points are selected by clicking SET REFPOS A and SET REFPOS B buttons respectively. See, e.g., FIG. 6.

A data retrieval function of the application program accesses location data provided to the application program and a scan controller function of the program controls scanning based on the accessed location data.

In one aspect, a micropositioner is provided to move a sensor relative to the microfluidic substrate. The micropositioner is moved from a location to a specific microchannel by using a MICROCHANNEL drop down list to select a microchannel to which the micropositioner is to be moved. A user selects the MOVE button on the display interface (FIG. 6) to initiate the system action which controls movement of a sensor positioned by the micropositioner to an appropriate microchannel outlet in the substrate at the maximum speed which has been inputted into the application. This feature may be used to control movement of a sensor in a sensor chamber to in front of an appropriate microchannel outlet which opens into the sensor chamber and/or to control movement of a sensor (e.g., a cell) from a recording well, a reservoir or treatment chamber in a microfluidic substrate to an appropriate microchannel. Additionally, or alternatively, the substrate can be moved relative to a stationary sensor. The microfluidic system can implement these system actions by sending signals to a microprocessor associated with either, or both, a stage on which the substrate is placed or a micropositioner coupled to the sensor.

In one aspect, the workstation comprises a probe such as a patch clamp pipette which additionally functions as a micropositioner. The application program can be used both to control the movement of the probe and to detect alterations of the sensor (e.g., to perform patch clamp recordings).

However, in yet another embodiment, the application program alters the movement of the sensor relative to a stationary probe by controlling the movement of a micropositioner (e.g., such as a holding pipette) stably associated with the sensor and/or by controlling the movement of the microfluidic substrate which contains the sensor within a sensor chamber.

In one embodiment, when a particular configuration is selected, the display interface displays a virtual representation of the substrate. For example, a particular geometry of microchannels on a substrate may be indicated on the display as a grid on which further system properties are indicated (e.g., such as distance between microchannels, numbers of microchannels, relationship to a sensor chamber, fluid reservoir or various other substrate components (e.g., valves, sensors, etc.) may be indicated. In one aspect, the position of the substrate relative to a scanning device (e.g., stage or micropositioner/probe) is inputted into the display interface. For example, the user interface will provide a series of selectable coordinates corresponding to microchannel positions or other features on substrate and selecting the coordinates or inputting the coordinates into the interface will identify a selected location on the substrate.

Preferably, the system supports at least three different modes of microchannel scanning. The basic procedure comprises: using the basic movement of the system to move a micropositioner to a microchannel selected as a start microchannel. The user selects the last microchannel by using the microchannel dropdown list. The display screen provides a dropdown list with at least three different scan modes to select from. Depending on the selected scan mode, different additional dropdown menus will be available. In one aspect, the user interface provides a series of selectable coordinates corresponding to microchannel positions or other features on substrate and selecting the coordinates or inputting the coordinates into the interface identifies a selected location on the substrate. The application program accesses the location data and sends instructions to external hardware for controlling scanning that directs the movement of the microfluidic substrate (e.g., via a stage on which the substrate is placed), the movement of one or more sensors (e.g., via a movable micropositioner), or the movement of both of these.

In one aspect, one of the system's scan mode comprises input trigger setting. In this mode, movement is controlled by external triggers from the external hardware. When an input trigger is detected, the system provides instructions to the external hardware to initiate a system action, i.e., such as moving the micropositioner from a first position to a position in front of a selected microchannel. The system may coordinate the system action of movement with another system action (such as detecting or recording). A user may also select an OUTPUT MICROCHANNEL TRIGGER action to be sent just before movement from one microchannel to the next.

In another aspect, a system scan mode comprises a CONTINUOUS MOVEMENT option. Preferably, a user inputs a parameter TIME PER MICROCHANNEL into the display of the user interface to select the time a micropositioner or probe will spend in proximity to the outlet of each microchannel. Maximum speed defaults may be set according to which stage hardware is identified for the application. In one aspect, a parameter “output start trigger” is provided to indicate the start of movement by the substrate and/or sensor. An “output microchannel trigger” can be checked to send triggers at each of the microchannels.

In a further aspect, the scan mode comprises alternating microchannel delays. In this mode, movement of the substrate is controlled by selecting EVEN MICROCHANNELS or ODD MICROCHANNELS. The parameter OUTPUT START TRIGGER is selected to indicate the start of movement of the substrate and/or the sensor to an appropriate start position. The system may also provide OUTPUT MICROCHANNEL TRIGGERS at each microchannel. In one aspect, where at least two microchannels lie in different planes, movement of the substrate may be mediated by selecting coordinates corresponding to the three-dimensional location of the microchannel and delays may be defined which are appropriate for microchannels at particular coordinates. A plurality of time settings (e.g., pause intervals or maximum speed for a scan may be selected for particular coordinate locations. In one aspect, the user interface displays a table or grid indicating the coordinates and by moving a cursor to appropriate coordinate(s), the user may program the movement of the substrate and/or sensor relative to the substrate.

Other substrate functions or processes may additionally be controlled by the application program in conjunction with other external hardware and/or with other programs in the suite. In one aspect, the application system communicates instructions to command various other system actions, such as: reagent addition; detection; fluidic movement; electroporation; electrophoresis; concentration; focusing; mixing; separation; cell movement; detection; patch clamp recording and the like.

Additionally, the application system also can communicate commands to provide coordination between two or more system actions. For example, substrate movement, movement of reagents, fluid, cell(s) or other sensors in the substrate is coordinated so that reagents are added to appropriate microchannels of the substrate and one or more cells/sensors are delivered to appropriate microchannel outlets for exposure to appropriate fluid streams.

In one aspect, the workstation provides a mechanism to programmably control fluid movement in one or more microchannels of the substrate. One or more fluid sources can be interfaced with the microfluidic system via connector tubing through which fluid flow may be controlled using switch relays and solenoid valves responsive to signals from a i/o module, preferably fitted with DC output modules to which the solenoid valves are connected. The output module is preferably connected to an analog/digital input/output card. Different valves may be selected using transister-based circuits in a digital i/o module to switch between different types of fluid (e.g., buffer containing or agent containing) in response to transistor transister logic signals from data acquisition card (e.g., such as a MacADIOS II card), e.g., interfaced to the fluid source via an i/o panel.

Valve outputs can be divided into two or more microchannels using tubing leading into separate manifolds. Solution can be made to flow from a constant-flow chamber flow into a single valve, where it diverges into two or more microchannels. Constant-flow chambers can comprise multiple output lines, each controlled by a separate valve and for flow into an industry standard microtiter plate in communication with the microfluidic substrate. Alternatively, or additionally, the contents of pre-filled microtiter plates can be delivered to appropriate reservoirs of a microfluidic substrate using positive pressure. In other aspects, a bank of pipet tips can deliver appropriate solutions to different reservoirs of the microfluidic substrate.

Information relating to the type of fluid being delivered is stored in the data acquisition program memory and in one preferred aspect, correlated to a detection event (e.g., correlating the response of one or more sensors to a particular type of fluid). The information can be provided as part of an identifier at the fluid source. For example, when a plurality of fluid streams are being delivered from a microtiter plate, the microtiter plate may comprise identifying information relating to the types of fluid in each well of the plate. The identifying information may be provided in the form of one or more barcodes, smartcards, or radio tags (e.g., such as manufactured by Irori).

In one aspect, fluid delivered through a microchannel to a sensor comprises an agent and different doses of the agent are provided from different fluid streams delivered by a plurality of microchannels having outlets in the sensor chamber. The fluid streams comprising agent may be interdigitated with fluid streams comprising buffer as described above.

As discussed above, movement of fluids in one or more microchannels of the microfluidic substrate may be controlled by pressure, using valves, by electric potential differences (e.g., provided through electrical elements plated onto the substrate), or by a combination thereof. The application of voltage and pressure may be controlled by microswitches in communication with the application program or with another program in the suite which can access information stored by the application program. In one aspect, fluid movement is coordinated with the movement of one or more sensors.

Electrical elements can also be used to control such processes as separation of molecules in one or more microchannels, focusing or concentration, mixing, movement of cells or other components in one or more microchannels, and the like.

Similarly, detector elements or sensors can be placed in one or more microchannels through which fluid flow to detect the presence of molecules or conditions (e.g., temperature, pH, etc.). The output of such detection events is preferably communicated to the application program or another program in the suite to which the application program has access. Preferably, the output is also communicated to the data acquisition system and can be displayed on the user interface in a suitable form (e.g., as text, as a graphical representation, or a combination thereof).

Sensor responses can be detected periodically or continuously.

In one particularly preferred aspect, the microfluidic system comprises one or more detectors for performing patch clamp recording. Preferably, detectors are coupled to amplifiers that are designed to handle multimicrochannel data and facilitate simultaneous recordings, e.g., from a plurality of sensors which are cells. The physiological responses of one or more cells/sensors to one or more fluid streams are recorded and stored in the memory of the data acquisition system. The recording system can include, but is not limited to, a digital recorder, a computer, volatile memory, involatile memory, a chart recorder or a combination of recording devices. In one preferred aspect, automated routines perform waveform analysis on each recording, e.g., using standard patch clamp recording software such as clampex/pulse. Electrophysiological traces from individual cells, when multiple cells are recorded, can be displayed in separate windows or superimposed for viewing and analysis. Routines within the data acquisition program can measure and analyze various electrical properties of the cell.

Preferably, the patch clamp software permits on-line signal analysis (e.g., i/v curves plot, amplitude histograms, spectral density, computations between traces), timers, automated command functions, programmable pulse generator specification, and data transfer to the data acquisition system. Preferably, the software when executed by a data processing system causes a user device to display an interface which allows visualization of digitised signals, automatic scaling, zoom and cursor movements, and enables on-line analysis of peaks, variance, late currents, maximum and minimum, rise time, time constant of exponential, area, slope, pulse duration, pulse voltage, values of cell capcitance, membrane conductance, access resistance, junction potential, and the like. Still more preferably, the software permits real-time data acquisition. Also, preferably, the software permits application-specific protocols to be stored for future use.

Microfluidic substrate components (e.g., valves, electrical elements, sensors, and the like) and macroscale components which interface with these may be controlled by functions which are part of the application program or part of a controller program which communicates with both the application program and data acquisition program. In one preferred embodiment, a controller program or a controller routine of an application program automatically obtains identifying data from signals sent by various elements of the external hardware without requiring user attention or input of this information. The substrate may also be identified by an identifier (e.g., a bar code or radio tag) that can identify the substrate to the controller program/routine which will then access data relating to the different components associated with the particular substrate.

In one aspect, some system actions are a system response to identifying one or more components on the substrate. For example, a particular pattern of fluid flow through different microchannels may be initiated on recognition of a particular type of substrate.

Function data relating to various components of the microfluidic system (whether microscale or macroscale) can be displayed on the display of the user interface. In one aspect, a single function is viewed at a time (e.g., in response to a user query or in response to selection of a component identifier displayed on the interface). In another aspect, a schematic of the substrate is displayed and a user can zoom in or enlarge a particular portion of the substrate and identify a system component whose function is to be displayed by the system.

Preferably, data acquisition and system functions are coordinated such that data received by the system (e.g., a response by a sensor such as a patch clamp recording) can be correlated with one or more functions and/or properties of the system. For example, in one aspect, sensor responses are correlated with data regarding the movement of the sensor relative to the microfluidic substrate, and in particular, relative to one or more microchannels on the substrate.

Preferably, the data acquisition program product comprises data management routines. For example, in one aspect, the data acquisition program product includes routines for searching and determining relationships between data structures (e.g., record files, tags) in the database. The data acquisition program product may be stored in the same memory as other programs in the suite or can be stored at a different location (e.g., accessed through a server or network). Preferably, the program product provides the ability to communicate results and records electronically.

In one embodiment of the invention, the data acquisition system comprises a search function which provides a Natural Language Query (NLQ) function. The NLQ accepts a search sentence or phrase in common everyday usage from a user (e.g., natural language inputted into an interface of the user interface of the system) and parses the input sentence or phrase in an attempt to extract meaning from it. In another embodiment of the invention, the search function recognizes Boolean operators and truncation symbols approximating values that the user is searching for. However, in another embodiment, the search query is communicated through the selection of options displayed on the user interface (e.g., after a detection event, such as a patch clamp recording). Search systems which can be used are described in U.S. Pat. No. 6,078,914.

The data acquisition program product preferably comprises a instructions enabling it to read codes, terms, or data inputted by the user into the interface, or received from the system itself (e.g., in the form of signals from the external hardware) and allows the user/system to access and display appropriate information from a relationship table in which data are stored. In one aspect, detection data are cross-referenced with data relating to other system properties (e.g., such as microchannel number, composition of fluid stream to which a sensor has been exposed, exposure time, etc) or other system functions.

The relationship determining function of the data acquisition program product can comprise any system known in the art, including, but not limited to regression, decision trees, neural networks, fuzzy logic, expert systems, and combinations thereof.

Methods of Using the System

The invention provides a method for programmably exposing a sensor to different solution environments in a microfluidic substrate. A user provides instructions to a system as described above by interfacing with the display screen of a user interface. The display screen displays a representation of the substrate (e.g., such as an image) and virtually marks start and stop points on the representation of the substrate using one or more joysticks or other input modules (e.g., such as a mouse or keyboard cursors). The user also communicates one or more substrate properties to the system, for example by inputting into a field a description of the property, selecting a radio button, checking a box and the like. Substrate properties include, but are not limited to: numbers of microchannels present on the substrate, numbers of reservoirs, numbers of sensor chambers, placement of reservoirs or chambers relative to microchannels, and the like.

In one aspect, a user is able to view microchannels in the substrate on the virtual representation of the substrate on the display of the user interface as a grid of columns and rows. The columns are preferably identified according to microchannel properties such as location (e.g., MICROCHANNEL-ID), BUFFER-SOLVENT-SAMPLE DESCRIPTION; TEMPERATURE; PRESSURE; VOLTAGE; and the like. In another aspect, the interface identifies system actions or system parameters such as WAIT-TIME, TIME-LOG-FOR-RECORD MATCHING, RECORD-or-Not-Record, SCANNING TRAVERSAL. System actions or parameters may be associated with values, such as an amount of time.

In one aspect, system instructions are associated with buttons for associating system actions (e.g., MOVE, SCAN) with system properties. For example, a user can mark a microchannel 1 on the interface and click the button “move” to perform the system action of moving a sensor in a sensor chamber in proximity to a microchannel 1 (the location of the microchannel being the system property). In another preferred aspect, a user clicks on a button to start movement of the sensor relative to a series of fluid streams exiting from the microchannels. A user selects the system property, for example, identifying the target microchannel to which the sensor is to be moved and a direction for microchannel scanning (e.g., FORWARD or REVERSE). Scanning can be implemented by moving the sensor relative to a stationary substrate, by moving the substrate relative to a stationary sensor or by moving the sensor and the substrate. The sensor will now traverse microchannels until either the first (or last) microchannel is reached, or until a selected microchannel is reached.

Preferably, one or more fluid streams from the microchannels provides a stream of an analyte, or ligand (e.g., an agonist or antagonist), a buffer or a cell for contacting to the sensor. The user can actuate a system action when the sensor is in suitable proximity to a microchannel providing a fluid stream of interest. In one preferred aspect, the system action performed is detection of the sensor's response to the fluid stream. In a particularly preferred aspect, when the sensor is a cell, the system action is a recording event, such as a clamp/pulse recording. Recording events may be brief (e.g., at each microchannel the sensor is exposed to) or long (during a scanning sweep as the sensor/cell is swept across a plurality of microchannels (e.g., from the first to last microchannel). Recording is preferably started by the user before the scanning movement is initiated. Detection events may be manually initiated by the user by selecting a detection button, e.g., RECORD.

While a system action is in progress, a user may push a stop button to stop the system action even if not completed. For example, the user may push a STOP button to interrupt a scanning action. Optionally, movement may halt at the next available microchannel, either for a preset amount of time, or for a default pause period of time. In one aspect, a halt interval is on the order of seconds. In another aspect, there is no halt interval, and a halt is followed, substantially immediately by another system action, such as a new scan in the same or a different direction. A detection event (e.g., a new recording event) can be initiated at the next target microchannel reached, or another system action, such as renewed scanning can be initiated.

In one aspect, the detection event will determine the next system action. For example, an analysis component of the data acquisition system may recognize values from one or more recording events as a trigger to send instructions to the external hardware via the application program to scan in a particular direction and/or at a particular rate of speed. In another aspect, if, after scans past a plurality of consecutive microchannels (e.g., about 5), no recorded signals, or unexpected signals are obtained, the application program will receive a trigger from the data acquisition system to send instructions to the scanning controllers (e.g., the external hardware operably coupled to the stage and/or a micropositioner/probe) to reverse and repeat a scan past selected channels and/or to move the sensor to new target channel(s) (e.g., delivering fluid streams which are expected to trigger a response by the sensor and/or which are not expected to trigger a response, e.g., such as a buffer-delivering channel).

The system preferably records the time of each system action for later review, e.g., such as for inspection and clampex/pulse integration purposes. In the embodiment where a detection event occurs as a sensor is scanned across a plurality of microchannels, the application program of the system inserts tags into the recorded data containing information about system properties (e.g., such as information about which microchannel, segments of the recording belongs to). Preferably, this information is visible on the display interface. Alternatively, data from the system action, such as data from the detection event log, will be inserted later into a recorded data file. If the current recording is a “multiple file recording”, the user can inspect the log data to select among the recorded files. In this aspect, the system memory comprises a database.

Preferably, the database is a relational database comprising a table of records of recording events, the records representing data acquired after scanning a sensor across one or more channels. Data in the database can be selected and displayed for viewing, analysis, and the like, e.g., by inputting queries into a display interface or by selecting options or links displayed on the display interface. Records may also be stored in separate files identified by individualized identifiers (e.g., such a numbers, 1,2, . . . n) and browsing can be implemented using a data management program as is known in the art.

Continuous System Action

In one aspect, a plurality of system actions is performed coordinately in response to programmed instructions in the system memory. For example, the user specifies a given wait-time in first row in a displayed grid that represents the microchannels and copies this value to all microchannels in the grid.

In one particularly preferred aspect, the system action comprises patch clamp recording and the user selects a configuration for a clampex/pulsefit program suitable for recording. The configuration typically comprises no external triggers, since the recording is initiated manually. The system is programmed to include a recording time long enough to include all microchannels. This may be determined initially by performing a manual scan using the tags stored in the system database to determine the appropriate time interval.

When a plurality of system actions are programmed, a single record file may be obtained for all microchannels. In one aspect, a continuous detection event (such as a patch clamp recording) is started manually. For example, a user manually initiates recording by executing a clampex/pulsefit program just before scanning is about to commence. The user sets the direction for scan motion and clicks the RECORD button and a scan which sweeps a sensor across preset number(s) and type(s) of microchannels is initiated.

Because scanning may be implemented by more than one component of the workstation (e.g., via a scanning stage, and/or micropositioner/probe), the application program can be used to rapidly expose a sensor to a plurality of changes in solution environment, the effect of which can be monitored by the data acquisition program. System versatility is enhanced still further because the stage and micropositioner/probe can be moved in an x-, y, z-, and circular motion. For example, the sensor may be moved up, down, forward, backward, at an angle and/or in an arc, by controlling the movement of the scanning stage and micropositioner/probe.

Intervals between system actions also can be pre-programmed. This application is useful when the microchannels contain alternating buffer-solvent-sample microchannels. The sensor waits in the buffer microchannels for a time period on the order of seconds or less. The system can be programmed to include a pause interval as small as about 30 msec in each buffer-solvent-sample microchannel. For example, in one aspect, a ‘wait-time’ is programmed for the two first microchannels in the grid, of a first time interval (e.g., about 2000 ms) for the first microchannel and a second time interval (e.g., about 30 ms) for the second microchannel. These values are copied into the rest of the grid representing the microchannels of the substrate. The system enables ‘tags’ to be inserted into the recorded data.

In one aspect, the system is used to perform high throughput patch clamp recordings in a microfluidic substrate. The system executes suitable patch clamp recording software (such as clampex/pulsefit) and the display of the user interface of the system displays a screen of recording parameters. An external trigger (such as input from the user) starts a recording run. The user inputs a recording time into a field on the interface which is long enough to include all microchannels. In one aspect, a single record file is obtained for all microchannels and stored in the database.

In a RAMP operation, a screen of the system program is displayed which provides a visual representation of the microfluidic substrate. Each microchannel in the substrate is represented as represented as a grid on the screen. The grid may be displayed with preselected information, for example, where the system properties (e.g., numbers and arrangement of microchannels) are used from in multiple experiments. Alternatively, a user may manually specify a grid (e.g., by inputting numbers of microchannels and inter-microchannel distance into fields in an initial display or by selecting a property value in a menu or list of values), after which a representation of the substrate conforming to the system properties identified is displayed.

Preferably, for all rows in the grid, the following data is specified or inputted:

1 . An Id of a microchannel is selected which matches the physical position order of microchannels in chip;

2. A time value is selected specifying the length of time to wait in a microchannel (i.e., specifying the length of a recording interval).

3. A RECORD or DON'T RECORD option is selected.

A configuration is selected from a recording display suitable for a particular run. In one aspect, the configuration includes instructions to: start recording in response to an external trigger. The time period for a detection event is selected which is matched to the wait time of a sensor (in a patch clamp system, a cell) in the microchannels. In some aspects, the wait time may be slightly longer than the detection time. For example, a user will select a RECORD option by clicking on a button, selecting from a menu of suitable time periods, or entering a value into a field. A suitable ramp protocol may also be provided to the system. Preferably, the system again provides a series of options for a user to select from. Multiple record files will be made, one for each trigger signal when there are multiple trigger signals. The user will set a direction for scan motion and click RECORD button.

Applications of the System

This invention exploits the potential for using microfluidic systems to control the delivery of a large number of different biologically active molecules and compounds (e.g., candidate drugs) to a sensor comprising a target molecule. Suitable molecules/compounds which can be evaluated include, but are not limited to, drugs; irritants; toxins; proteins; polypeptides; peptides; amino acids; analogs and modified forms of proteins; polypeptides, peptides, and amino acids; antibodies and analogs thereof; immunological agents (e.g., such as antigens and analogs thereof, haptens, pyrogens, and the like); cells (e.g., such as eukaryotic cells, prokaryotic cells, infected cells, transfected cells, recombinant cells, bacteria, yeast, gametes) and portions thereof (e.g., cell nuclei, organelles, secretogogues; portions of cell membranes); viruses; receptors; modulators of receptors (e.g., agonists, antagonists, and the like); enzymes; enzyme modulators (e.g., such as inhibitors, cofactors, and the like); enzyme substrates; hormones; metabolites and analogs thereof; nucleic acids (e.g., such as oligonucleotides; polynucleotides; fibrinotides; genes or fragments, including regulatory sequences, and/or introns, and/or coding regions; allelic variants; RNA; antisense molecules, ribozymes, nucleotides, aptamers), including analogs and modified forms thereof; metal clusters; and inorganic ions.

Combinations of two or more of any of these molecules also can be delivered, sequentially or simultaneously, to one or more sensors in the sensor chamber. Compounds also can be obtained from synthetic libraries from drug companies and other commercially available sources known in the art (e.g., including, but not limited, to the LeadQuest® library comprising greater than 80,000 compounds, available through http://www.tripos.com/compounds/; ChemRx Diversity Library, comprising 1000 to 5000 compounds per scaffold, available through http://www.chemrx.com; the Nanosyn Pharma library, available through Nanoscale Combinatorial Synthesis Inc., Menlo Park, Calif., and the like) or can be generated through combinatorial synthesis using methods well known in the art. In aspects in which molecules are delivered to cells, any of the molecules described above may be taken up by cells by transiently exposing the cells to an electric field (e.g., in a cell treatment chamber or in a sensor chamber which is adapted for electroporation) as described above.

Providing Periodically Resensitized Ion Channel Sensors

Binding a compound (such as an agonist or modulator or drug) to a broad range of ion channels not only evokes conformational changes in these channels, allowing a flux of ions across a cell membrane, but also causes the ion channel to desensitize, i.e., to reside in a long-lasting, ligand-bound, yet shut-off and non-conducting state (see, e.g., Jones and Westbrook, 1996, GL Trends Neurosci. 19: 96-101). Desensitization of many types of ion-channels usually occurs within a few milliseconds and is thought to be one of the mechanisms by which synaptic information in the central nervous system is processed and modified. Densitization also may serve as a negative feedback mechanism that prevents excitotoxic processes caused by excessive activation of ion channels by neurotransmitters or other neuromodulators (see, e.g., Nahum-Levy, et al., 2000, Biophys J. 80: 2152-2166; Swope, et al., 1999, Adv. Second Messenger Phosphoprotein. Res. 33: 49-78).

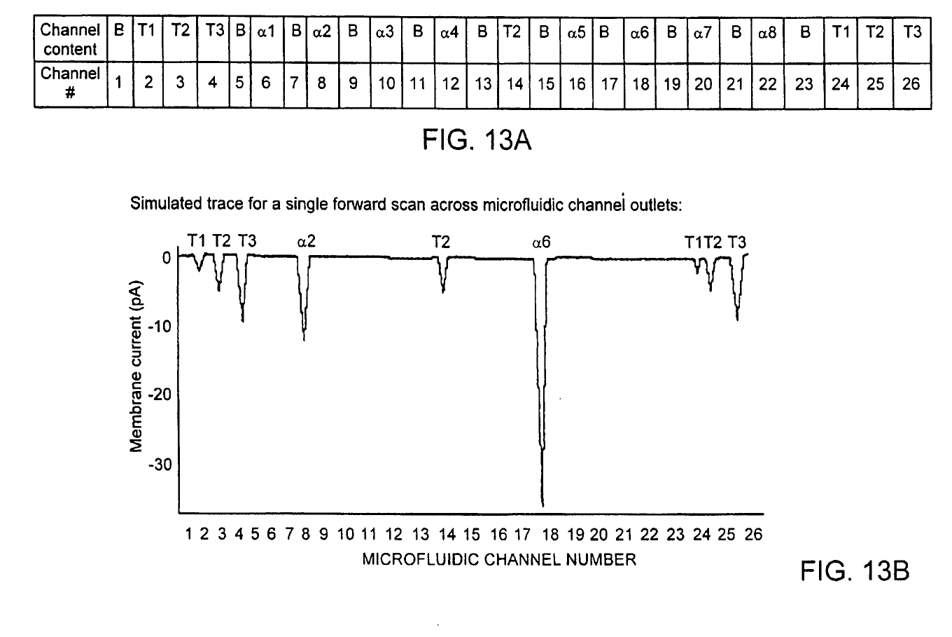

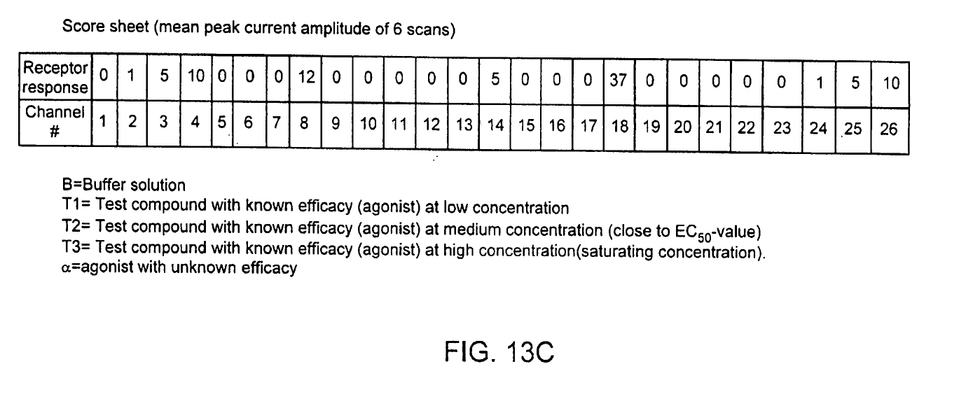

In one aspect, to achieve high screening rates in, for example, HTS applications, patch-clamped cell(s) in the sensor chamber are moved from the outlet of one microchannel to the next in rapid succession. To achieve rapid resensitizaton of ion channels and receptors, microchannels delivering samples comprising suspected modulators, agonists, or drugs of receptor/ion channels are interdigitated with microchannels delivering buffer for resensitization of the receptor/ion channels (e.g., buffer free of any agonist). In addition to resensitizing ion channels and receptors, this delivery of buffer onto cells between ligand and drug exposure serves to wash out ligands and drugs previously administered to the cell. Thus, in this aspect, the system is used to screen for an agonist or modulator or drug of a specific ion-channel by providing a periodically responsive ion channel sensor. For example, by providing pulsed or steady-state flow delivery of buffer to the sensor, the system provides a cell that is resensitized when exposed to a microchannel outlet delivering a candidate agonist or modulator or drug. FIGS. 13A-C show simulated screenings of unknown agonists according to one method using a microfluidic chip comprising 26 outlets feeding into a sensor chamber. The contents of each microchannel are shown in FIG. 13A. Agonists with known pharmacological action (e.g., known efficacy, or potency) have been included in certain microchannels to serving as internal controls or standards. The score sheet for this experiment, i.e., the patch clamp response obtained for each microchannel is shown in FIG. 13C.

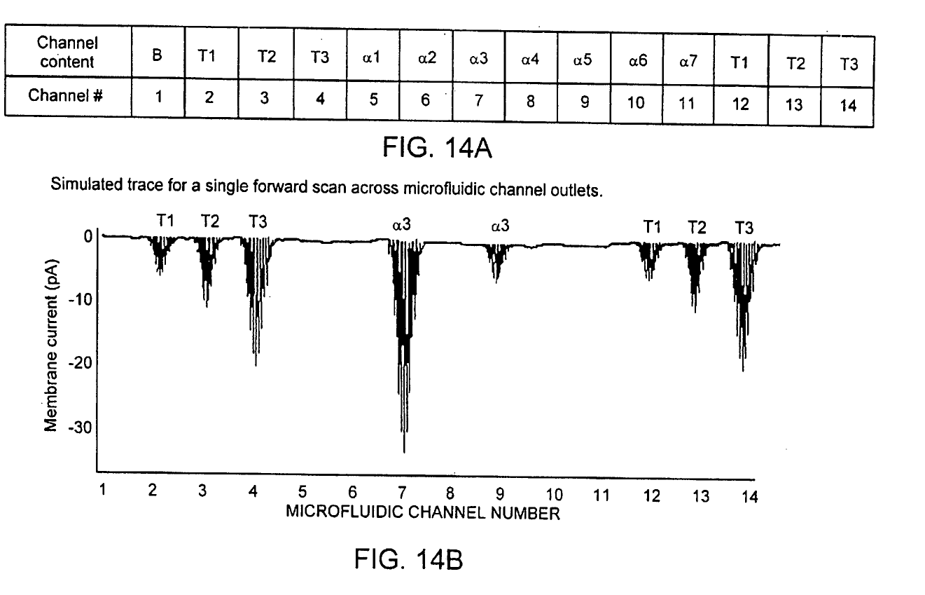

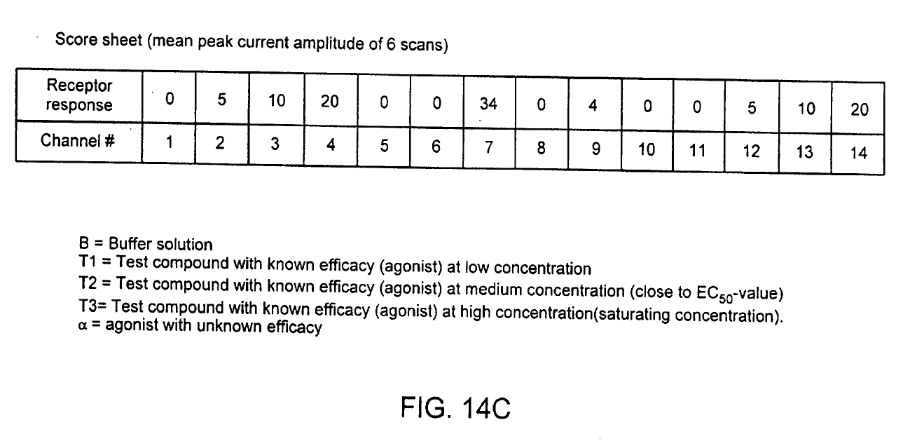

In another embodiment, an additional superfusion pipette proximal to the patch-clamped cell, e.g., in an arrangement that is adjacent to or coaxial with respect to the patch pipette (as detailed below), is used to continuously resensitize/wash receptors/ion channels on the cell surface. This enables cells to be extremely rapidly resensitized and washed (e.g., within ms) and enables several different readings/registrations of ion channel activation to be made as a cell moves across a microchannel outlet. FIGS. 14A-C show a simulated method of rapid resensitization used for screening of agonists which combines the use of a microfluidic chip comprising 14 outlets feeding into a sensor chamber with pulsed superfusion of agonist-free buffer solution using a fluidic microchannel (or micropipet) placed coaxial or orthogonal or otherwise in close proximity to a patched-clamped cell. The contents of each microfluidic microchannel are shown in FIG. 14A. Agonists with known pharmacological action (e.g., known efficacy, or potency) have been included in certain microchannels to serve as internal standards or test compounds. The simulated trace, shown in FIG. 14B, for a linear, single, forward scan of a cell-based biosensor across microfluidic microchannel outlets, show a plurality of peak responses obtained per single microchannel outlet. The score sheet for this experiment, i.e., the patch clamp response obtained for each microchannel is shown in FIG. 14C. In this case, a Gaussian-distributed response is obtained because it was modelled that the ligands exiting microchannels into the open volume had a gaussian distribution. Many other types of distributions can be obtained depending on substrate geometry and experimental parameters, such as level of collimation of flows. However, this type of repeated superfusion of cells during their passage across a single microchannel outlet allows dose-response information and high signal-to-noise ratios to be obtained for receptors/microchannels that rapidly desensitize.

To obtain desired data, variable scan rates of cell(s) across individual streams of sample and buffer and variable pressure drops across each microchannel can be implemented by the system, either from pre-programmed instructions or in response to feed-back signals from a detector in electrical communication with the patch clamp electrode (e.g., based on a detected signal or in real-time).

The system thus can be used to change microenvironments rapidly around a cell comprising a receptor/ion-channel. For example, the system can provide a periodically responsive ion channel. Because of the small dimensions of the substrates and microchannels used herein, which allows for rapid mass transport, the system enables a user to screen for drugs at the rate of hundreds per second (i.e., millions per hour) using one patch clamp sensor, provided drugs and resensitization solutions are delivered sequentially at a comparable rate to the sensor. As discussed above, scanning rates can be modified to account for the physiological responses of a cell-based sensor, e.g., providing slower scanning rates for receptors that equilibrate slowly.

Generating Dose-Response Curves and Analyzing Ion-Microchannel Pharmacology

Dose-response curves provide valuable information regarding the actions and potencies of drugs. Obtaining dose-response curves using traditional methods involving micropipettes often can be time consuming and tedious. The present invention, which uses microfluidics for the rapid and controlled manipulation of the microenvironment around cell(s), is uniquely suited for dose-response measurements. Dose-response relationships most often follow a sigmoidal curve in a lin-log plot, and can be described by the Hill logistic functions:

I=Imax/[1+(EC50/C)n]

Where I is the whole-cell current, C is the concentration of ligands, Imax is the maximal current (i.e., when all microchannels are in the open state), EC50 is the half-maximal value (i.e., when half of the receptor population is activated, and often equals KD, the dissociation constant of the ligand), and n is the Hill coefficient that reflects the stoichiometry of ligand binding to the receptor.

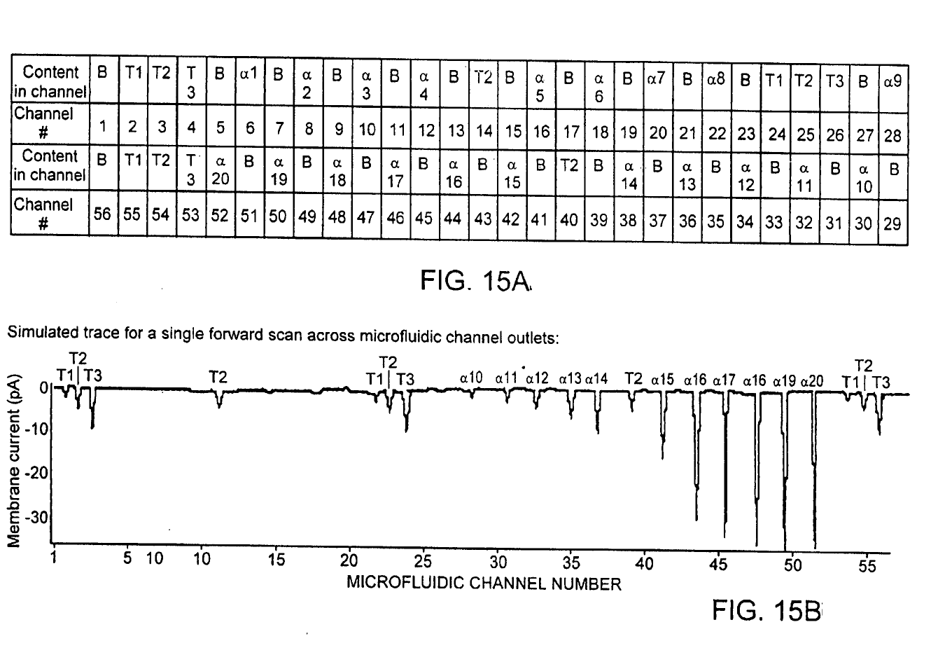

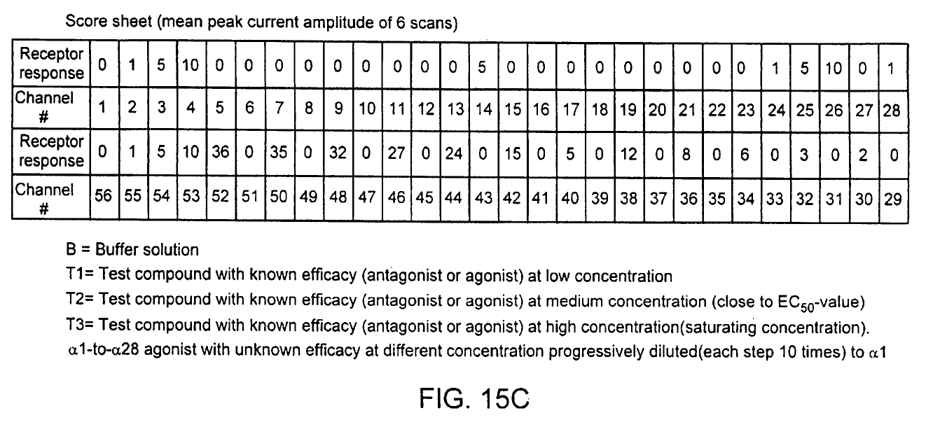

In one aspect, to achieve dose-response information for agonists, patch-clamped cell(s) in the sensor chamber are moved from the outlet of one microchannel to the next in rapid succession. Microchannels delivering agonists at different concentration are interdigitated with microchannels delivering buffer free of agonist (e.g., to resensitize receptors/ion channels and/or to wash out compounds previously administered to the cell, as described above). Preferably, the serially or sequentially diluted agonists are loaded into different microchannels. FIG. 15A is an example of such a loading scheme in a 56-microchannel substrate. Agonist is present at highest concentration in microchannel 52 and then is serially diluted at each subsequent microchannel until microchannel 6. Agonists with known pharmacological action (e.g., known efficacy, or potency) have been included in certain microchannels to serve as internal standards. Preferably, the agonist concentration from the microchannel with the highest concentration to the microchannel with the lowest concentration covers many orders of magnitude. FIG. 15B show simulated patch clamp recordings of agonists at different concentration as described above. From the score sheet for this simulated experiment, i.e., the patch clamp response obtained for each microchannel as shown in FIG. 15C, a dose-response curve can be constructed.

Similarly, with some modifications, dose-response curves can be obtained for antagonists as well using the system which is described in more detail below. Furthermore, as described above, the combination of microfluidics with patch clamp can provide a wide range of information about the actions of modulators on ion-channels, e.g., such as the association and dissociation constants of a ligand for its receptor, and whether a modulator is an agonist or an antagonist of a receptor. It is also possible, however, to obtain dose-response information from accumulated responses of ligands without washing or resensitizing the receptors with interdigitated flows of buffer. In this aspect, the microchannels need only contain ligand solutions at different concentrations.

(i) Detection and Characterization of Agonists

Partial Agonists

The ability of a drug molecule to activate a receptor is a graded property, rather than an all-or-nothing property. If a series of chemically related agonists acting on the same receptor are tested on a cell, the maximal response (i.e., the largest response that can be produced by an agonist in high concentration) generally differs from one agonist to another. Some compounds (known as “full agonists”) can produce a maximal response whereas others, referred to “partial agonists”, can only produce a submaximal response. Some partial agonists can even act as inhibitors when they reach a certain concentration level. Thus, by using a defined ion-channel together with a known agonist that produces a maximal response, the grade of an agonist's activity can be monitored (see, e.g., FIG. 13A-C).

(ii) Detection and Characterization of Antagonists

In one aspect, the system is used to screen for antagonists of ion-microchannel activity. Suitable ion-channels which can be evaluated include: (i) ion channels that do not de-sensitize; (ii) ion-channels that desensitize (iii) ion-channels that desensitize but which mediate large current fluctuations when activated; and (iv) ion-channels whose desensitizing property is blocked by irreversible binding of an allosteric modulator (e.g., such as a lectin). To detect antagonists, the ion-channels or receptors expressed by a biosensor need to be activated or “tested” by an agonist during, before, or after, application of the antagonist. For example, different antagonists can be applied together with a well-defined agonist with known pharmacological properties. Antagonists at different concentrations also can be loaded into microchannels together with agonists at a constant concentration.

To achieve rapid resensitizaton of ion channels and receptors, microchannels containing agonist and antagonist (e.g., such as ligands and drugs) are interdigitated with microchannels delivering buffer free of any agonist or antagonist (e.g., buffer for resensitization of the receptor/ion channels). In addition to resensitizing ion channels and receptors, exposure of cells to buffer between periods of exposure to ligands and drugs serves to wash out ligands and drugs previously administered to the cell. Thus, in this aspect, the system is used to provide a periodically responsive ion channel sensor. Antagonists are detected in this system by their inhibition of the agonist-induced response.

In another aspect, the system is used to screen for antagonists which can be detected through attenuation in the signal mediated by constantly pre-activated receptors/ion-channels. In this particular setup, different channels are loaded with different antagonists, or with the same antagonist at different concentrations, or a combination of both, while each channel comprising antagonist comprises agonist at a constant concentration. To achieve continuous activation of receptors and ion channels, microchannels containing agonist and antagonist are interdigitated with microchannels delivering buffer and agonist at the same concentration as in the microchannels supplemented with antagonist. This delivery of buffer supplemented with agonist onto cells between ligand and drug exposure serves to wash out ligands and drugs previously administered to the cell and also can serve to resensitise a receptor/ion channel.

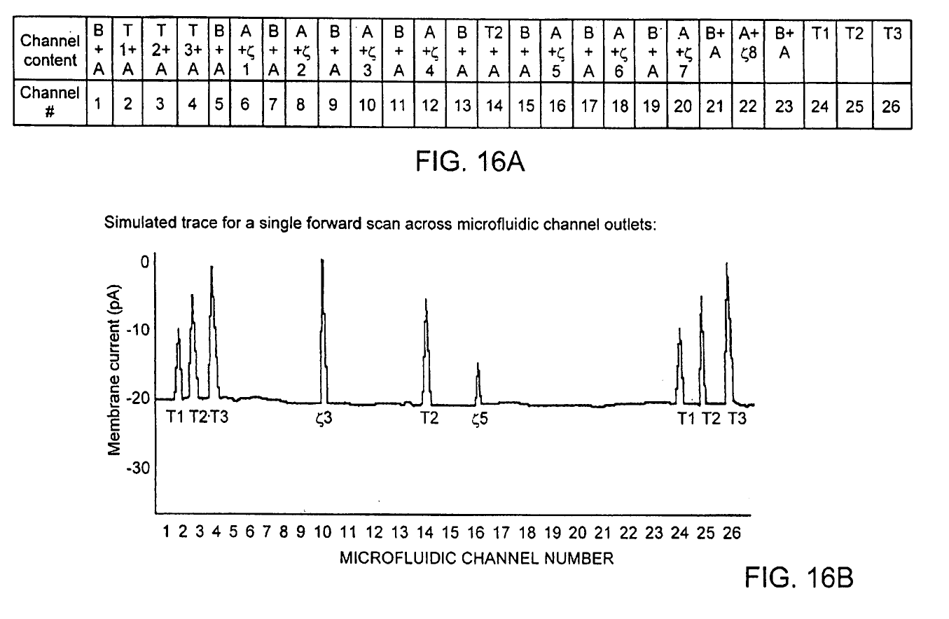

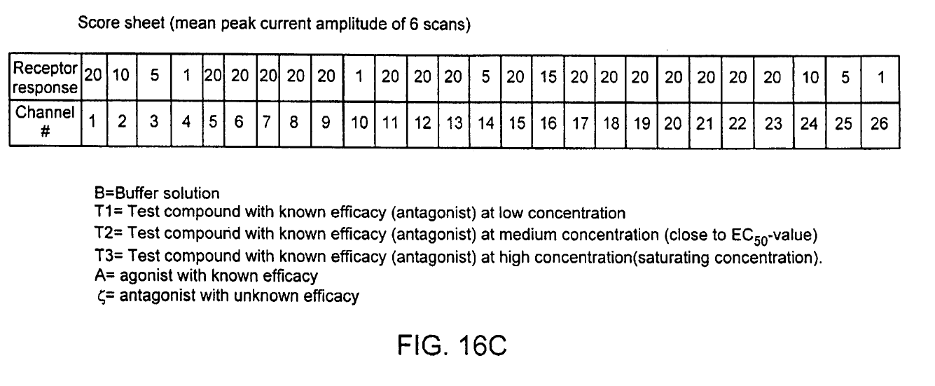

A simulation of such an experiment is shown in FIGS. 16A-C. The contents of each microchannel is shown in FIG. 16A. Antagonists with known pharmacological action (blocking potency) have been included in certain microchannels to serve as internal standards. The simulated trace shown in FIG. 16B represents a linear single forward scan of a cell-based biosensor across microfluidic microchannel outlets. As shown in the Figure, a plurality of peak responses are obtained per single microchannel outlet. The score sheet for this experiment, i.e., the patch clamp response obtained for each microchannel, is shown in FIG. 16C, from which the antagonist with the highest blocking potency can be identified.

Competitive Antagonism

This type of antagonism refers to competition between agonists and antagonists at the same binding site on the receptor. Reversible competitive antagonism is characterized by a shift in the slope of a dose response curve to higher concentrations while maintaining the same maximum response and the slope of the curve. In irreversible competitive antagonism, no change in antagonist occupancy is observed when the cell is exposed to agonist.

Non-Competitive Antagonism

Non-competitive antagonism describes the situation where the antagonist blocks, at some point, the chain of events that leads to the production of a response by the agonist. In this type of antagonism, the agonist and antagonist either bind to different sites on the receptor/ion channel or the antagonists simply block the ion channel pore. The net effect is to reduce the slope and maximum of the agonist's dose-response curve.

Isosteric Inhibition

This type of antagonism refers to the self-inhibition of agonists above a certain concentrations; that is, an agonist will start to antagonize its own action at a sufficiently high concentration. A bell-shaped dose-response curve often signals the presence of this kind of antagonism.

Detection of Modulators of Presynaptically Expressed Ion-Channels

In another aspect, the system is used to detect a modulator of a presynaptically expressed ion-channel. Strategies for studying presynaptically localized ion-channels often include patch clamp recordings of synaptosomes (i.e., pinched-off nerve terminals produced by homogenizing brain tissue) inserted in proteoliposomes or planar phospholipid bilayers (see, as described in Farley and Rudy, 1988, Biophys. J. 53: 919-934; Hirashima and Kirino, 1988, Biochim Biophys Acta 946: 209-214, for example). The method of Hirashima and Kirino, 1988, supra, is particularly preferred, as it is a simple and rapid technique for generating giant proteoliposomes comprising presynaptically expressed ion-channels which can be used as biosensors for patch clamp analysis in the system according to the invention.

Detection of Ligands Acting on Orphan Receptors/Ion-Microchannels

Conventional drug discovery approaches often are initiated by the discovery of ligand's biological activity which is subsequently used to characterize its tissue pharmacology and physiological role. Typically, after the ligand is characterized, the corresponding receptor is identified as target for drug screening in HTS applications. A relatively novel strategy for characterizing orphan receptors (i.e., receptors with an undefined biological activity) is often referred to as a “reverse pharmacology” approach. The reverse approach starts with an orphan receptor of unknown function that is used as target for detection of its ligand. The ligand is then used to explore the biological and pathophysiological role of the receptor. High-throughput screening is initiated on the receptor at the same time that the ligand is being biologically characterized in order to develop antagonists that will help determine the therapeutic value of the receptor.

The present invention is particularly useful for a reverse pharmacological approach. In one aspect, the system comprises a cell-based biosensor which is a non-native cell line which expresses an exogenous orphan receptor (e.g., such as an ion channel). Suitable native cell lines, include, but are not limited to, HEK-293, CHO-KI, and COS-7. There are several benefits coupled to screening ion channels in a non-native cell background. First, a transfected cell line containing a null background (e.g., which does not ordinarily express the orphan receptor) allows one to be certain of the molecular identity of the gene responsible for the observed signal. Second, the orphan receptor can be over-expressed, thus improving the signal-to-noise of the screening read-out. Third, host cells with low background conductances can be chosen to allow very sensitive assays of certain types of ion channels. Finally, these cell lines are relatively easy to culture and are robust enough to be handled by automated screening systems.

Detection of Modulators of Neurotransmitter Vesicular Release

Patch-clamp techniques to measure membrane capacitance, developed over ten years ago (see, e.g., Neher and Marty, 1982, Proc. Natl. Acad. Sci. U SA 79: 6712-6716), provide a powerful tool to study the underlying mechanism and control of exocytosis.

The surface area of a cell depends on the balance between exocytosis and endocytosis. Exocytosis results in the discharge of vesicle contents (i.e., such as neurotransmitters) into the extracellular space and the incorporation of vesicle membrane into the plasma membrane, leading to an increase in cell surface area. During endocytosis, parts of the plasma membrane are retrieved, resulting in a decrease in the surface area. Changes in net exocytotic and endocytotic activity thus can be monitored by measuring changes in cell surface area.

Membrane capacitance is an electrical parameter of the cell that is proportional to the plasma membrane area. Thus, providing the specific capacitance remains constant, changes in plasma membrane area resulting from drug-induced modulation of exocytotic and endocytotic activity through presynaptically located ion-microchannels, can be monitored by measuring membrane capacitance by means of patch clamp in the open sensor chamber of the system.

Determining Permeability Properties of a Cell

When a cell used in a screening procedure expresses a broad range of ion-channel types, characterizing the ion permeability properties of the cell's activated ion-channels can be used to characterize a drug's interaction with the cell. Information about permeability properties of an ion-channel can be determined by monitoring reversal potential which can be determined by evaluating current-to-voltage relationships, created from measurements of agonist-evoked currents at different holding potentials. By employing the reversal potential and knowledge about intra- and extra-cellular ion concentrations, the relative ion-channel permeability properties are determined from different models.

Noise Analysis of Current Traces

Analysis of current-traces from ion-channels activated by agonists can be performed on both an ensemble- and single-channel level for further characterization of an agonist-ion-channel interaction. The Fourier transformation of the autocorrelation function obtained for the total current recorded with whole-cell patch clamp yields power spectra that can be fitted by single or double Lorentzian functions. These fits provide information about mean single-channel conductances and ion-channel kinetics (e.g., mean single channel open time) through analysis of the frequency dependence of the current response (i.e., corner frequency). In principle, although a more difficult and tedious technique, recordings obtained from outside-out patch-clamp configurations also can be analysed to measure single-channel opening intervals and different conductance levels mediated by the same receptor-ion channel complex.

### EXAMPLES

The invention will now be further illustrated with reference to the following examples. It will be appreciated that what follows is by way of example only and that modifications to detail may be made while still falling within the scope of the invention.

### Example 1

Microfabrication of a Substrate

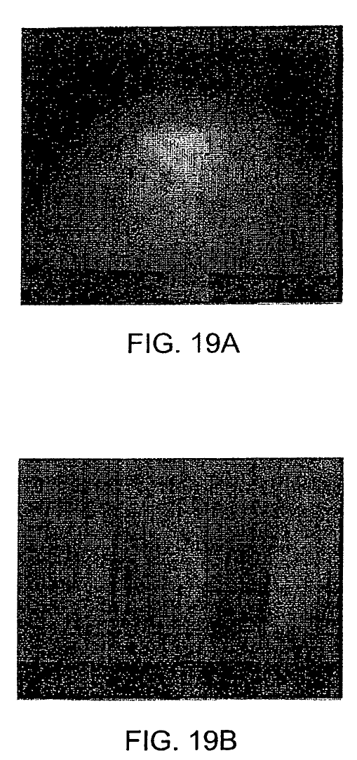

FIG. 19 shows examples of microchannels fabricated in silicon by deep reactive ion etching in SF6. Masks for photolithography were produced using standard e-beam writing on a JEOL JBX-5DII electron beam lithography system (medium reflective 4″ chrome masks and Shipley UV5 resists, 50 keV acc. voltage, dose 15 μC/cm−2, exposure current 5 nA). The resist was spin coated at 2000 rpm for 60 s giving 250 nm of resist and soft baked for 10 minutes at 130° C. on a hotplate before exposure. The pattern was post exposure baked for 20 minutes in an oven at 130° C. and developed for 60 s in Shipley MF24-A, rinsed in DI water and etched in a reactive ion etcher (Plasmatherm RIE m-95, 30 s, 50 W, 250 mTorr, 10 ccm O2). The chrome was etched for 1-2 minutes in Balzers chrome etch #4, the mask was stripped of the remaining resist using Shipley 1165 remover and rinsed in acetone, isopropanol and DI water. A 3″, [100], two sides polished, low N-doped Silicon wafers with 700 nm of thermally grown silicon dioxide and a total thickness of 380 μm was cleaned in a reactive ion etcher Plasmatherm RIE m-95 (30 s, 50 W, 250 mTorr, 10 ccm O2), spin coated with Shipley S-1813 photoresist at 4000 rpm, giving 1.3 μm of resist, and exposed for a dose of

110 mJ/cm−2 at 400 nm wavelength on a Carl Süss MA6 mask aligner. The wafer was developed for 45 s in Shipley MF319 rinsed in DI water and ashed in a reactive ion etcher (Plasmatherm RIE m-95, 30 s, 50 W, 250 mTorr, 10 ccm O2). The wafer was hard baked for 10 minutes at 130° C., the silicon dioxide was etched with SioTech buffered oxide etch and rinsed in DI water. The wafer was stripped of the remaining resist with acetone, rinsed in isopropanol and DI water. The other side of the wafer was spin coated with Shipley AZ4562 photoresist at 3000 rpm for 30 seconds giving approximately 8 μm of resist, soft baked for 3 minutes at 100° C. on a hotplate and exposed for a dose of 480 mJ/cm−2 at 400 nm wavelength on a Carl Süss MA6 mask aligner. The pattern was developed for 200 seconds in Shipley MF312 and DI water in 50:50 mix, rinsed in DI water, and ashed in a reactive ion etcher (Plasmatherm RIE m-95, 30 seconds, 50 W, 250 mTorr, 10 ccm O2). The pattern defined in the photoresist AZ4562, the recording chamber and the combined access holes and sample wells was etched in a STS Multiplex deep reactive ion etcher using SF6 as etching gas and C4F8 as passivation gas at 600 W of RF power and 30 W of platen power. The system was operating at a constant APC angle of 74% and the etching time was 12 seconds with an overrun time of 1 second, and the passivation time 8 seconds with an overrun time of 1 second. The etching rate was approximately 4.9 μm/minute and the etching time 60 minutes resulting in a depth of approximately 300 μm. The wafer was stripped of the remaining resist in acetone, rinsed in isopropanol and DI water. The pattern in silicon dioxide defining the microchannels was etched with the same system as before but with 800 W of RF power, at a constant APC angle of 68% and the etching time was 7 s with an overrun time of 0.5 s, and the passivation time 4 second with an overrun time of 1 second. The etching rate was approximately 3.3 μm/min and the etching time 30 minutes resulting in a depth of 100 μm. The wells and the recording chamber were completely etched through resulting in holes in the wafer at these points. The microchannels were sealed to a 3″, 1000 μm thick wafer of Corning #7740 borosilicate glass using anodic bonding at a temperature of 450° C. and a voltage of 1000 V. The maximum current during bonding was typically 500 μA.

### Example 2

Re-sensitization of Patch-Clamped Cell Using Microfluidic-Based Buffer Superfusion and Cell Scanning

Microchannels were molded in a polymer, polydimethylsiloxane (PDMS), which were then sealed irreversibly onto a glass coverslip to form an enclosed microchannel having four walls.

The procedure used is the following:

(1) A silicon master used for molding PDMS was fabricated by first cleaning the wafer to ensure good adhesion to the photoresist, followed by spin coating a layer (˜50 μm) of negative photoresist (SU 8-50) onto the wafer. This layer of negative photoresist was then soft baked to evaporate the solvents contained in the photoresist. Photolithography with a mask aligner was carried out using a photomask having the appropriate patterns that were prepared using e-beam writing. The exposed wafer was then baked and developed by washing away the unexposed photoresist in an appropriate developer (e.g. propylene glycol methyl ether acetate).

(2) This developed wafer (master) was surface passivated by silanizing in vacuo with a few hundred microliters of tridecafluoro-1,1,2,2-tetrahydrooctyl-1-trichlorosilane for a few hours.

(3) Degassed PDMS prepolymer was poured on top of the silicon master and left in an oven to cure at 60° C. for two hours, (4) The cured PDMS mold containing the microchannel features was then sealed irreversibly to a glass substrate after oxidization in an oxygen plasma for ˜1 min. Microchannel dimensions we used in this example were approximately 100 μm wide and 50 μm deep.

The experiments described here used a simple single-microchannel structure. This microchannel was interfaced to a polyethylene tubing by first punching a smooth hole through the PDMS with a sharp hole-puncher having the appropriate dimensions. Polyethylene tubing having an outer diameter slightly greater the punched hole was inserted into the hole, and the tubing formed a pressure seal owing to the elastomeric nature of PDMS. The polyethylene tubing was connected to a syringe needle having the appropriate size (gauge), which was connected to a syringe. Controlled pressure for driving fluid flow was accomplished with a high precision syringe pump (CMA/100, Microinjection pump, Carnegei Medicin).

Patch clamp experiments were carried out in the whole-cell configuration. The pipettes for whole-cell recording were fabricated from thick-walled borosilicate glass capillaries having an outer diameter of 1.5 mm and an inner diameter of 0.86 mm (Harvard Apparatus LTD Edenbridge, Kent, UK). The diameters and the resistances of the tips were ˜2.5 μM and 5-15 MΩ, respectively. The estimated series resistance was always <50 MΩ and holding potentials were corrected for voltage errors due to series resistance. The patch clamp electrode solution contained 100-mM KCl, 2-mM MgCl2, 1-mM CaCl2, 11-mM EGTA, and 10-mM HEPES; pH was adjusted to 7.2 with KOH. All experiments were performed at room temperature (18-22° C.).

Signals were recorded with an Axopatch 200 A (Axon inc. California, U.S.A) patch-clamp amplifier, at a holding potential of −70 mV, and were digitized and stored on the computer hard drive (sample frequency 10 kHz, filter frequency 200 Hz using a 8 pole Bessel filter) and analyzed using a PC and Clampfit 8.1 software (Axon inc.). The experimental chamber containing the microchannel structure was mounted on an inverted microscope stage equipped with 40× and 10× objectives (Nikon, Japan). Mounted to the microscope was a CCD camera (Hamamatsu) connected to a video for recording of the scan rates, the sampling rate for the video was 25 Hz. This equipment together with micromanipulators (Narishigi, Japan) was placed on a vibration-isolated table inside a Faraday cage. The patch clamp amplifier, the Digidata board, filters, the video and PCs, were kept outside the cage to minimize interference from line frequency.

Adherent PC-12 cells were cultivated on circular cover slips in Petrie dishes for 2-6 days (DMEM/F12 medium supplemented with antibiotics and antimyocotin (0.2%), fetal calf serum (10%), and L-glutamine). Before the patch clamp experiments, cells were washed and detached in a HEPES-saline buffer, containing (in mM): 10 HEPES, 140 NaCl, 5 KCl, 1 CaCl2, 1 MgCl2, 10 D-glucose (pH 7.4), and placed in the open buffer reservoir at the outlet of the microchannel.

The strength of the seals was tested with cells that were patched-clamped without entering into a whole-cell configuration. A membrane holding potential of −70 mV was applied and the cell was positioned 10 μm away from the microchannel outlet. Different flow rates, which varied between 0.3-21 mm/s, were applied while the seal was continuously monitored. The patched seal was stable (no shift in the current trace) for flow rates up to 6.7 mm/s, in this particular experiment.

For the re-sensitization experiment, agonist was added to the open reservoir where the cell was patched while buffer was delivered from the syringe into the microchannel and exits the microchannel into the open reservoir. The patch-clamped cell was placed ˜10 μm away from the outlet of the microchannel. The reservoir in which the patch-clamped cell resides was filled with 1 mM acetylcholine (agonists). Buffer was delivered by the syringe pump into the microchannel and was continuously flown through the microchannel at ˜3 mm/s.

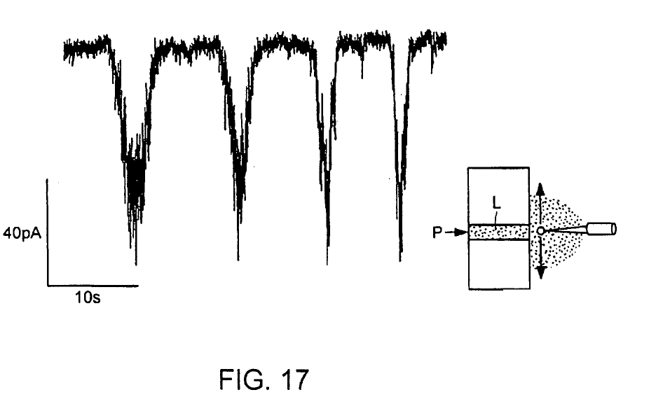

No current was observed while the giga Ohm seal was stable (5-20 Gohm) as the cell was moved, in a direction parallel to the microchannel, from ˜10 μm to ˜80 μm from the outlet of the microchannel. This fact means the patch-clamped cell was superfused by the buffer exiting from the microchannel and thus was not in contact with the agonists in the open reservoir. At ˜80 μm from the outlet of the microchannel, the patched cell was scanned repeatedly at ˜100 μm/s, in a direction perpendicular to the microchannel, between the reservoir containing agonists and the microchannel outlet (FIG. 17).

De-sensitization of the current response could be observed after exposure to the agonist for longer periods of time (>5 s) as a decrease of the mean whole-cell current. No de-sensitization of the cells was seen for the shorter exposure times (<5 s) to the agonist nor for repeatedly short exposures as long as the patched cell was re-sensitized in agonist free buffer between each exposure.

### Example 3

Rapid Scanning of a Patch-Clamped Cell Across Interdigitated Streams of Ligands and Buffer for HTS Applications

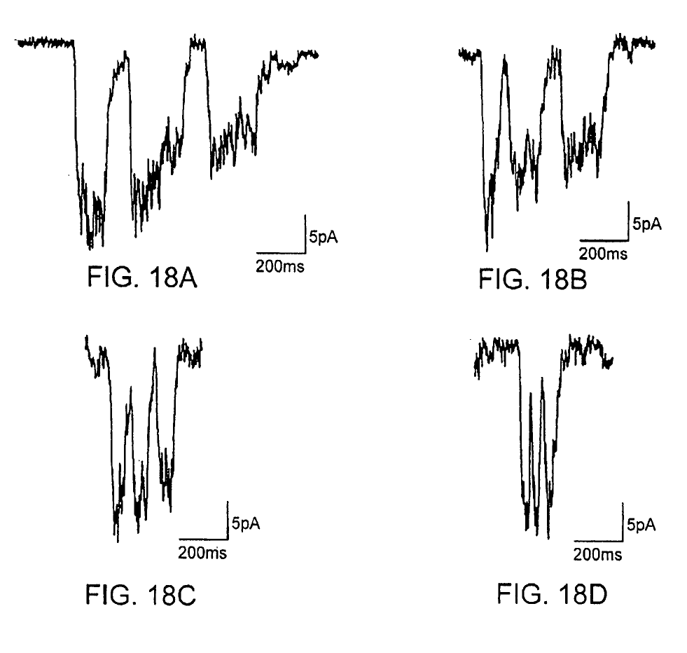

One preferred embodiment for implementing HTS using the current invention is to scan a patch-clamped cell rapidly across interdigitated streams of buffer and ligands, with each ligand stream corresponding to a different drug. In these applications, as discussed above, both the flow rate of the fluids exiting the microchannels and the scan rate of the patch clamped cell are important. FIGS. 18A-D show the response of patch-clamped whole cells after being scanned across the outlets of a 7-microchannel structure. The width of each microchannel is 100 μm, the thickness is 50 μm, and the intermicrochannel spacing is 25 μm. The procedure used for fabricating the microchannels and for patch clamping are identical to that described in Example 2 (see above). The patch clamped cell used was a PC-12 cell, which was placed between 10 to 20 micrometers away from the outlets of the microchannels. Microchannels 1, 3, 5 and 7 were filled with PBS buffer, while microchannels 2, 4 and 6 were filled with acetylcholine. The flow rate of the fluid streams was 6.8 mm/s.

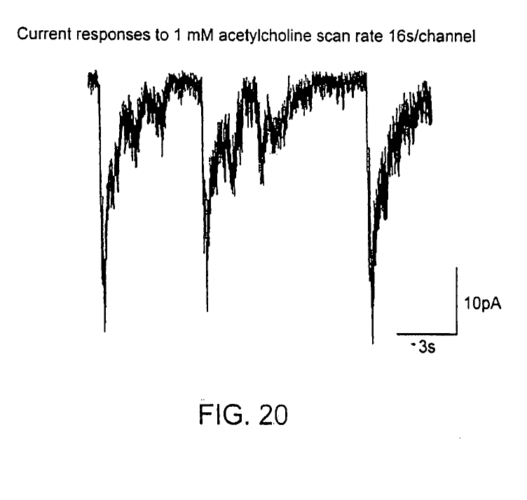

In FIGS. 18A-D, a patch-clamped cell was scanned across interdigitated streams at four different scan rates: A, 0.61 mm/s; B, 1.22 mm/s; C, 2 mm/s; and D, 4 mm/s. The difference in the scan rate is reflected in the width of the whole cell current response peaks, the wider the width, the longer the transit time and the wider the peak width. In addition, for slow scan rates (e.g., FIG. 18A), the maximal response for each peak decreases as the patch-clamped cell is scanned from one acetylcholine stream to the next. This decrease in the peak response is caused by desensitisation of the patch-clamped cell as a result of the slow scan rate that led to a longer residence time for the cell in the acetylcholine stream. From FIG. 18A, it can seen the decrease in height from the second to third peak is greater when compared to the decrease from the first to second peak. This is consistent with the fact that the longer residence time (i.e., larger peak width) of the patch-clamped cell in the second stream causes more desensitisation. As the scan rate increases (FIGS. 18C and 18D), the residence time in the acetylcholine stream decreases and desensitisation is no longer an issue. For fast scan rates (e.g., tens of ms) as shown, for example, in FIG. 18D, no desensitisation can be detected. FIG. 20 shows the opposite scenario in which the scan rate is slow (seconds), and desensitisation is pronounced as the patch-clamped cell is scanned across the width of the acetylcholine stream. From these experiments, it is clear that controlling the scan rate is critical for achieving optimal performance of the system for HTS applications. Scanning rates can be controlled by any of the mechanisms described above or by other methods known in the art.

Data obtained by the system relating to the dynamics of desensitisation and re-sensitization can be exploited to provide useful information in elucidating ion-microchannel pharmacology, kinetics and identity.

### Example 4

Dose-Response Measurements by Rapid Scanning of a Patch-Clamped Cell Across Interdigitated Streams of Buffer and Ligands Having Different Concentrations

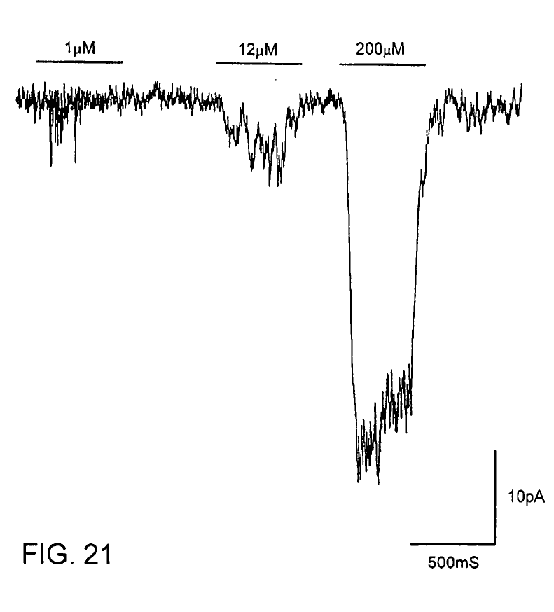

The microchannel structure and experimental setup used in Example 3 can be used to carry out dose-response measurements, in which the concentrations of the ligands in each of the ligand streams differ by predetermined amounts. FIG. 21 shows the result of one such experiment, in which three different concentrations (1 μM, 12 μM and 200 μM) of nicotine were applied to a patch-clamped cell. In a 7-microchannel structure, microchannels 1, 3, 5 and 7 were filled with PBS buffer, whereas microchannels 2, 4, and 6 were filled with 1 μM, 12 μM, and 200 μM nicotine, respectively. The flow rate used was 3.24 nm/s and the cell-scanning speed was 250 μm/s. The patch-clamped cell was placed between 10 to 20 μm away from the outlet of the microchannel.

At 1-μM concentration of nicotine, the whole-cell current response was barely discernible in the patch-clamp trace. The current peak for 12 μM was detected with good signal-to-noise ratio, and the peak that corresponds to 200 μM was approximately 15 to 20 times that of the peak for 12-μM. With these measurements, a dose-response curve can be generated that provides valuable information about drug action and ion-microchannel pharmacology. It should be emphasized that a number of on-chip techniques for gradient generation as well as off-chip methods for preparing different concentrations of ligands can be used (see, e.g., Dertinger, et al., 2001, Analytical Chemistry 73: 1240-1246). In addition, the number of different concentrations used for constructing dose-response curves will in most cases be greater than that used in this example, and will depend on the required concentration resolution and range desired for a particular application.

Variations, modifications, and other implementations of what is described herein will occur to those of ordinary skill in the art without departing from the spirit and scope of the invention. The publications, patents, applications and other references cited herein are all incorporated by reference in their entirety herein.

What is claimed is:

1. A computer program product comprising:
a computer readable medium having computer readable program code embodied in the medium for causing an application program to execute on a computer,
wherein the program product comprises instructions for controlling one or more functions of a microfluidic substrate in response to received data regarding one or more substrate properties.

2. A computer program product comprising a computer readable medium having computer readable program code embodied in the medium for causing an application program to execute on a computer,
wherein the program product comprises instructions for controlling one or more functions of a microfluidic substrate in response to received data regarding one or more properties of a sensor in fluid communication with at least one microchannel of the substrate and optionally, for controlling one or more functions of the microfluidic substrate in response to received data regarding one or more substrate properties.

3. A computer program product comprising a computer readable medium having computer readable program code embodied in the medium for causing an application program to execute on a computer,
wherein the program product comprises instructions for controlling one or more functions of a microfluidic substrate, including instructions for controlling scanning of a sensor relative to an outlet of at least one microchannel in the substrate.

4. The computer program product of any of claims 1-3, wherein the one or more functions comprises scanning a sensor relative to an outlet of at least one microchannel in the substrate by moving the sensor, moving the substrate, moving both the sensor and the substrate and/or by varying pressure at at least one microchannel.

5 The computer program product of claim 4, wherein the one or more functions comprises scanning a sensor relative to a plurality of microchannels.

6. The computer program product of claim 5, wherein scanning is continuous.

7. The computer program product of claim 5, wherein scanning is interrupted by one or more programmed pauses during a selected time interval.

8. The computer program product of any of claims 1-3, wherein the one or more fictions is selected from the group consisting of: the movement of fluid in at least one microfluidic channel of the substrate; the movement of a cell in at least one microfluidic channel of the substrate; the delivery of an agent to at least one channel in the substrate; the movement of an agent in at least one channel in the substrate; scanning a sensor relative to an outlet of at least one microchannel in the substrate by moving the sensor; moving the substrate, moving both the sensor and the substrate; varying pressure in at least one microchannel; separation of molecules and/or ions in at least one channel of the substrate, concentration of molecules in at least one channel of the substrate; mixing; heating; focusing; detection; electroosmosis; electrophoresis; electroporation; electroinjection, electrofusion, recording electrical properties of a sensor in fluid communication with the at least one channel; changes in fluid pressure; and combinations thereof.

9. The computer program code according to claim 8, wherein the one or more functions comprises scanning a sensor relative to an outlet of at least one channel in the substrate and wherein the computer program product comprises a computer readable program code for coordinating scanning with data acquisition.

10. The computer program product of any of claims 1-3, wherein the computer program product further comprises a computer readable program code for causing a computer to input data received from a detector in proximity to the sensor.

11. The computer program product of claim 10, wherein the data comprises signal data relating to a response or reaction of the sensor.

12. The computer program product of claim 11, wherein the response or reaction is selected from the group consisting of a physiological response, a change in Calcium levels, hybridization, binding, change in electrical properties, introduction of an agent into and/or onto a cell, introduction of an agent into an intracellular compartment, a change in electrical properties of a cell, and combinations thereof.

13. The computer program product of claim 12, wherein the one or more functions comprises delivering an agent to the sensor and wherein the signal data comprises data relating to a response or reaction of the sensor to the agent.

14. The computer program product of any of claims 1-3, wherein the one or more functions comprises delivering an agent to the sensor, and wherein the computer program product comprises a computer readable program code for coordinating delivery of the agent with data acquisition.

15. The computer program product of any of claims 1-3, wherein the microfluidic substrate further comprises at least one reservoir and/or cell chamber in communication with at least one channel of the substrate.

16. The computer program product of claim 15, wherein the one or more functions comprises delivery of a fluid and/or agent from the channel to the reservoir and/or cell chamber.

17. The computer program product of claim 15, wherein the one or more functions comprises exposure of the at least one channel, reservoir and/or cell chamber and or a sensor in the at least one channel, reservoir and/or cell chamber to an electric field.

18. The computer program product of claim 17, and wherein the computer program product comprises a computer readable program code for causing a computer to input data relating to electric field properties.

19. The computer program product of claim 2 or 3, wherein the one or more functions comprises delivering an agent to the sensor, and wherein the computer program product comprises a computer readable program code for causing a computer to input data relating to electric field properties and/or a response or reaction to the agent.

20. The computer program product of any of claims 1-3, wherein the one or more functions comprises delivering an agent to at least one channel of the substrate and wherein the computer program product comprises a computer readable program code for causing a computer to input data relating a parameter of the agent.

21. The computer program product of claim 20, wherein the parameter is selected from the group consisting of: name of agent, amount of agent; a property of the agent, a previous response of a sensor to the agent, and combinations thereof.

22. The computer program product of claim 20, wherein the agent is delivered from the at least one channel to a sensor and wherein the computer program code further comprises instructions for generating output data relating to the response of a sensor to the agent.

23. The computer program product of claim 22, wherein the output data comprises a dose-response data.

24. The computer program product of any claims of 1-3, wherein the computer program product further comprises instructions for execution by a processor in communication with a fluid delivery control mechanism which controls delivery of fluid through at least one channel of the substrate.

25. The computer program product of claim 24, wherein the fluid delivery control mechanism controls the delivery of streams of buffer and agent through channels of the substrate.

26. The computer program product of claim 24, wherein the fluid delivery control mechanism controls the delivery of fluid streams selected from the group consisting of: streams of different doses of agonists; streams of different doses of antagonists; streams of one or more agonists, streams of one or more antagonists, streams of buffer, and combinations thereof.

27. The computer program product of any of claims 1-3, wherein the computer program product further comprises instructions for execution by a processor in communication with a pressure control mechanism to vary pressure in at least one channel of the substrate.

28. The computer program product of claim 27, further comprising instructions for execution by a processor in communication with an agent delivery control mechanism to deliver of an agent through at least one channel of the substrate.

29. The computer program product of claim 28, wherein the instructions comprise instructions to deliver different agents to at least two channels of the substrate.

30. The computer program product of claim 28, wherein the instructions comprise instructions to deliver different amounts of agents to at least two channels of a substrate.

31. The computer program product of claim 28, wherein the instructions comprise instructions to change an amount of agent delivered to at least one channel of a substrate.

32. The computer program product of claim 28, wherein the instructions comprise instructions to deliver buffer through at least one channel of the substrate.

33. The computer program product of claim 28, wherein the instructions comprise instructions to deliver buffer through a channel adjacent to at least one channel delivering an agent.

34. The computer program product of claim 2 or 3, wherein the computer program product further comprises instructions to superfuse the sensor with buffer at selected time intervals, and wherein the instructions include a providing delay between acquisition of data relating to a response or reaction of the sensor and exposure to the buffer.

35. The computer program product according to any of claims 1-3, wherein the computer program product further comprises a memory, storing data relating to substrate properties.

36. The computer program product according to claim 2 or 3, wherein the computer program product comprises a memory storing data relating to properties of the sensor.

37. The computer program product of claim 36, wherein the properties of the sensor comprise reactions or responses of the sensor.

38. The computer program product according to any of claims 1-3, wherein the computer program product comprises a memory storing data relating to parameters of functions of the microfluidic substrate.

39. The computer program product of any of claims 1-3, wherein the substrate property is selected from the group consisting of: number of channels in the substrate, channel geometry, distance between channel outlets; distance between channel inlets, position of one or more sensors relative to channel outlets; position of the substrate relative to a scanning device; the position of at least one channel of the substrate; position of a substrate relative to a sensor; substrate material, substrate temperature; and combinations thereof.

40. The computer program product of any of claims 1-3, wherein the computer program product comprises instructions to alter a parameter of the one or more functions in response to a measured condition of the microfluidic substrate or a sensor in communication with at least one channel of the microfluidic substrate.

41. The computer program product of claim 40, wherein the measured condition is selected from the group consisting of: arrival of an analyte, agent, and/or cell at a channel outlet, arrival of an analyte, agent, and/or cell at a microchannel inlet, fluid movement through the at least one channel, an electroporation event, an electrophoresis event, a concentration event, a separation event, a mixing event, a recording event, pressure, a change in pressure, fluid velocity, a change in fluid velocity, a parameter of an electric field, and combinations thereof.

42. The computer program product of claim 40, wherein the substrate function comprises scanning and the parameter of the function is selected from the group consisting of: the number of microchannel outlets to be scanned, time to complete scanning, length of pauses at microchannel outlets, speed of scanning, trajectory of scanning, maximum speed of scanning, alternating channel delay, continuous movement, scanning in response to a selected input received from a sensor, pressure at at least one channel, fluid velocity in at least one channel and combinations thereof.

43. The computer program product of any of claims 1-3, wherein the computer program product further comprises instructions for a processor in communication with a macroscale device which is in communication with the microfluidic substrate.

44. The computer program product of claim 43, wherein the macroscale device is selected from the group consisting of: a power supply, a pump head, pump, degasser, flow meter, injector manifold, a fluid delivery system, an agent delivery system, a pressure sensor; flow cell; concentration manifold, a cartridge, a fitting, a connector, a switch, a valve, a septum, a mixer, a compressor, an ultrasonic bed, an extractor, a focusing device, a dialysis chamber, an absorption chamber, a metabolite chamber, a toxicity chamber, a cell chamber, a detector, an RFID tag, a reagent vessel, a separation column, a focusing column, a size exclusion column, an ion-exchange column; affinity column, a mass spectrometer, a solid-phase extraction bed, a filter, a sieve; a frit, a depth filter, a heater, a heat exchanger, a cooler; a magnetic field generator; electroporation device, electroinjector, microinjector, nanoinjector, a patch clamp pipette, a micropositioner, a micromanupulator, a microscope stage, a signal amplifier, a light source, and combinations thereof.

45. The computer program product of claim 43, wherein the instructions relate to an operation of the macroscale device on the microfluidic substrate.

46. The computer program product of claim 45, wherein the operation comprises delivery of fluid, an agent, a cell, pressure, a voltage, a current, ultrasound, light, and/or a radiofrequency to a region on the substrate.

47. The computer program product of claim 46, wherein the region comprises a channel, a reservoir or a cell chamber.

48. The computer program product of claim 46, wherein the operation comprises separation of molecules or ions in a fluid to be delivered to or from a channel, reservoir or cell chamber in the substrate.

49. The computer program product of claim 48, wherein the molecules or ions are selected from the group consisting of: proteins, polypeptides, peptides, nucleic acids, organic molecules, inorganic molecules, carbohydrates, metabolites, positive ions, negative ions, and combinations thereof.

50. The computer program product of claim 45, wherein the operation comprises heating or cooling of a fluid to be delivered to a channel in the substrate.

51. The computer program product of claim 45, wherein the operation comprises moving the substrate or a component of the substrate.

52. The computer program product of claim 51, wherein the component of the substrate comprises a sensor.

53. The computer program product of claim 45, wherein the operation comprises exposing a region of the substrate to light from a light source, and wherein the light source is a laser in optical communication with a sensor in a channel, reservoir and/or cell chamber of the substrate.

54. The computer program product of any of claims 1-3, wherein the computer program product further comprises computer program code for generating and displaying a graphical user interface.

55. The computer program product of claim 54, wherein the graphical user interface displays a screen on which at least one substrate property, parameter of substrate function, parameter of the macroscale device function, and/or property of a sensor in communication with the substrate, is displayed.

56. The computer program product of claim 54, wherein the graphical user interface displays a screen comprising fields for inputting data relating to the at least one substrate property, parameter of substrate function, parameter of the macroscale device function, and/or property of a sensor in communication with the substrate.

57. The computer program product of claim 54, wherein the function parameter is selected from the group consisting of the number of microchannel outlets to be scanned, time to complete scanning, length of pauses at microchannel outlets, speed of scanning, maximum speed of scanning, the trajectory of scanning and combinations thereof.

58. The computer program product of claim 57, wherein the trajectory of scanning is linear, non-linear or a combination thereof.

59. The computer program product of claim 54, wherein the graphical user interface displays a screen providing selectable options for a plurality of different scan modes for moving the substrate relative to a sensor, moving a sensor relative to a substrate, and/or varying pressure in at least one microchannel.

60. The computer program product of any of claims 1-3, wherein the computer program product comprises a data acquisition program embedded in a computer readable medium.

61. The computer program product of claim 60, wherein the data acquisition program comprises a search function, a relationship determining function, and/or a data retrieval function.

62. The computer program product of any of claims 1-3, wherein the computer readable medium further comprises a memory comprising data relating to scanning a sensor across one or more fluid streams of the microfluidic substrate and/or varying pressure at one or more microchannels of the microfluidic substrate.

63. The computer program product of claim 62, wherein the data relating to scanning the sensor comprises data relating to the number of microchannel outlets scanned, the time to complete a scan, pause time intervals at one or more channels, a property of a fluid stream delivered by one or more microchannel outlets, pressure at one or more microchannels, fluid velocity in one or more microchannels, data relating to the sensor response at one or more microchannel outlets, data relating to the trajectory of scanning and combinations of such data.

64. The computer program product of claim 63, wherein the property of the fluid stream comprises an identity or property of an agent in the fluid stream.

65. The computer program product of claim 2 or 3, wherein the sensor is a cell, a cell fraction, an organelle, a membrane comprising an ion channel, a receptor, a nucleic acid, a protein, a polypeptide, a peptide, small molecule, a drug, a chemical compound, a compound library, gene chip, protein chip, a surface plasmon energy sensor; an FET sensor; an ISFET; an electrochemical sensor; an optical sensor; an acoustic wave sensor; a sensor comprising a sensing element associated with a Quantum Dot particle; a polymer-based sensor; a single molecule or an array of molecules immobilized on a substrate.

66. The computer program product of claim 64, wherein the agent is selected from the group consisting of a drug; irritant; known toxin; candidate toxin; know carcinogen; candidate carcinogen; known mutagen; candidate mutagen; protein; polypeptide; peptide; amino acid; antibody; antigen binding molecules; antigen; hapten; pyrogen; cytokine; growth factor; cell; cell fraction; organelle; secretogogue; virus; viral particle; receptor; a modulator of receptor; ligand; enzyme; enzyme modulator; enzyme substrate; hormone; metabolite; nucleic acid, nucleotide, nucleobase; sugar; carbohydrate, small molecule; metal; ion; and analogs and modified forms thereof.

67. The computer program product of any of claims 1-3, further comprising instructions for managing, searching, mining, organizing, comparing, and/or representing data.

68. A microfluidic workstation comprising a data acquisition system for executing a computer program product of any of claims 1-3, wherein the data acquisition system is operably linked to the microfluidic substrate.

69. The workstation of claim 68, wherein the system provides data to a processor in communication with the substrate to alter one or more substrate functions.

70. The workstation of claim 69, wherein the system is pre-programmed to alter one or more substrate functions.

71. The workstation of claim 69, wherein one or more substrate functions is altered in response to feedback from the substrate.

72. The workstation according to claim 69 wherein the one or more substrate functions is selected from the group consisting of: the movement of fluid in at least one microfluidic channel of the substrate; the movement of a cell in at least one microfluidic channel of the substrate; the delivery of an agent to at least one channel in the substrate; the movement of an agent in at least one channel in the substrate; scanning a sensor relative to an outlet of at least one microchannel in the substrate by moving the sensor, moving the substrate, moving both the sensor and the substrate, or by varying pressure at at least one channel of the substrate; separation of molecules and/or ions in at least one channel of the substrate, concentration of molecules in at least one channel of the substrate; mixing; heating; focusing; detection; electroosmosis; electrophoresis; electroporation; electroinjection, electrofusion, recording electrical properties of a sensor in fluid communication with the at least one channel; changes in fluid pressure; and combinations thereof.

73. The workstation of claim 68, wherein the workstation comprises a data processing system comprising a memory.

74. The workstation of claim 73, wherein the data processing system accesses data from one or more computer program products, and wherein the data relates to properties or functions of the microfluidic substrate and/or properties of a sensor in fluid communication with at least one channel of the microfluidic substrate.

75. The workstation of claims 73 or 74 wherein the data processing system accesses data through one or more external databases.

76. The workstation of claim 68, wherein the system further comprises a sensor in fluid communication with at least one channel of the microfluidic substrate.

77. The workstation of claim 68, wherein the substrate comprises an identifier that can identify the substrate to a computer program product for accessing data relating to substrate properties and/or functions.

78. The workstation of claim 68, wherein the workstation further comprises a macroscale device.

79. The workstation of claim 78, wherein the macroscale device is selected from the group consisting of: a power supply, a pump head, pump, degasser, flow meter, injector manifold, a fluid delivery system, an agent delivery system, a pressure sensor; flow cell; concentration manifold, a cartridge, a fitting, a connector, a switch, a valve, a septum, a mixer, a compressor, an ultrasonic bed, an extractor, a focusing device, a dialysis chamber, an absorption chamber, a metabolite chamber, a toxicity chamber, a cell chamber, a detector, an RFID tag, a reagent vessel, a separation column, a focusing column, a size exclusion column, an ion-exchange column; affinity column, a mass spectrometer, a solid-phase extraction bed, a filter, a sieve; a frit, a depth filter, a heater, a heat exchanger, a cooler; a magnetic field generator; electroporation device, electroinjector, microinjector, nanoinjector, a patch clamp pipette, a micropositioner, a micromanupulator, a microscope stage, a signal amplifier, a light source, and combinations thereof.

80. The workstation of claim 78, wherein the macroscale device is in communication with a processor that receives instructions from the computer program product.

81. The workstation of claim 81, wherein the macroscale device comprises a detector for detecting a reaction or response of a sensor exposed to a fluid delivered by the substrate.

82. The workstation of claim 81, wherein the data acquisition system receives input data from the detector relating to the reaction or response.

83. The workstation of claim 82, wherein the input data comprises signal data relating to a response or reaction of the sensor.

84. The workstation of claim 83, wherein the reaction or response is selected from the group consisting of: a physiological response, a change in calcium levels, hybridization, binding, change in electrical properties, introduction of an agent into and/or onto a cell, introduction of an agent into an intracellular compartment, and combinations thereof.

85. The workstation of claim 69, wherein the one or more functions is executed in response to acquisition of data by the data acquisition system.

86. The workstation of claim 82, wherein the data acquisition system performs one or more operations on the data and executes the one or more functions when a predefined result of the one or more operations is obtained.

87. The workstation of claim 69, wherein the microfluidic substrate further comprises at least one reservoir and/or cell chamber in communication with at least one channel of the substrate.

88. The workstation of claim 69, wherein the one or more functions comprises delivery of a fluid and/or agent from the at least one channel to the reservoir and/or cell chamber.

89. The workstation of claim 69 or 88, wherein the one or more functions comprises exposure of the at least one channel, reservoir and/or cell chamber and or a sensor in the at least one channel, reservoir and/or cell chamber to an electric field.

90. The workstation of claim 89, wherein the data acquisition system receives input data from the workstation relating to properties of the electric field.

91. The workstation of claim 89, wherein the data acquisition system provides output data to a processor in communication with a power supply for generating the electric field.

92. The workstation of claim 91, wherein the output data comprises instructions for changing one or more properties of the electric field.

93. The workstation of claim 68, wherein the substrate comprises at least one channel and the workstation further comprises an agent delivery system for delivering one or more agents to at least one channel of the substrate.

94. The workstation of claim 68, wherein the substrate comprises at least one electrically conducting surface for delivering an electric field to a sensor in fluid communication with the at least one channel.

95. The workstation of claim 94, wherein the sensor comprises a cell structure and the electric field is of a strength sufficient to electroporate a membrane of the cell structure.

96. The workstation of claim 95, wherein the computer program product comprises a computer readable program code for causing a computer to input data relating to electric field properties and/or a response or reaction of the sensor to an agent.

97. The workstation of claim 69, wherein the one or more functions comprises delivering an agent to at least one channel of the substrate and wherein the computer program product comprises a computer readable program code for causing a computer to input data relating a parameter of the agent.

98. The workstation of claim 97, wherein the parameter is selected from the group consisting of: name of agent, amount of agent; a property of the agent, a previous response of a sensor to the agent; and combinations thereof.

99. The workstation of claim 97, wherein the agent is delivered from the at least one channel to a sensor and wherein the computer program code further comprises instructions for generating a output data relating to the response of the sensor to the agent.

100. The workstation of claim 68, wherein the workstation further comprises a fluid delivery mechanism for controlling delivery of fluid through at least one channel of the substrate and wherein the computer program code further comprises instructions for execution by a processor in communication with the fluid delivery control mechanism to control delivery of fluid.

101. The workstation of claim 100, wherein the instructions comprise instructions to deliver a plurality of agents to one or more channels of the substrate.

102. The workstation of claim 101, wherein the instructions comprise instructions to deliver different agents to at least two channels of the substrate.

103. The workstation of claim 101, wherein the instructions comprise instructions to deliver different amounts of agents to at least two channels of a substrate.

104. The workstation of claim 100, wherein the instructions comprise instructions to change an amount of agent delivered to at least one channel of a substrate.

105. The workstation of claim 100, wherein the instructions comprise instructions to deliver buffer through at least one channel of the substrate.

106. The workstation of claim 102, wherein the instructions comprise instructions to deliver buffer through a channel adjacent to at least one channel delivering an agent.

107. The workstation of claim 68, wherein the data acquisition system comprises a memory for storing data relating to at least one substrate property.

108. The workstation of claim 68, wherein the data acquisition system comprises a memory for storing data relating to parameters of functions of the microfluidic substrate.

109. The workstation of claim 107, wherein the at least one substrate property is selected from the group consisting of: number of channels in the substrate, channel geometry, distance between channel outlets; distance between channel inlets, position of one or more sensors relative to channel outlets; position of the substrate relative to a scanning device; the position of at least one channel of the substrate; position of a substrate relative to a sensor; substrate material, substrate temperature; and combinations thereof.

110. The workstation of claim 69, wherein the substrate function comprises scanning and the parameter of the function is selected from the group consisting of: the number of microchannel outlets to be scanned, time to complete scanning, length of pauses at microchannel outlets, speed of scanning, maximum speed of scanning, alternating channel delay, continuous movement, scanning in response to a selected input received from a sensor, trajectory of scanning, pressure at at least one microchannel, and combinations thereof.

111. The workstation of claim 68, wherein the computer program product further comprises instructions for a processor in communication with a macroscale device that is in communication with the microfluidic substrate.

112. The workstation of claim 111, wherein the macroscale device is selected from the group consisting of: a power supply, a pump head, pump, degasser, flow meter, injector manifold, a fluid delivery system, an agent delivery system, a pressure sensor; flow cell; concentration manifold, a cartridge, a fitting, a connector, a switch, a valve, a septum, a mixer, a compressor, an ultrasonic bed, an extractor, a focusing device, a dialysis chamber, an absorption chamber, a metabolite chamber, a toxicity chamber, a cell chamber, a detector, an RFID tag, a reagent vessel, a separation column, a focusing column, a size exclusion column, an ion-exchange column; affinity column, a mass spectrometer, a solid-phase extraction bed, a filter, a sieve; a frit, a depth filter, a heater, a heat exchanger, a cooler; a magnetic field generator; electroporation device, electroinjector, microinjector, nanoinjector, a patch clamp pipette, a micropositioner, a micromanupulator, a microscope stage, a signal amplifier, a light source, and combinations thereof.

113. The workstation of claim 111, wherein the instructions relate to an operation of the macroscale device on the microfluidic substrate.

114. The workstation of claim 113, wherein the operation comprises delivery of fluid, pressure, a voltage, a current, and/or a radiofrequency to a region on the substrate.

115. The workstation of clam 114, wherein the region comprises a channel, reservoir or cell chamber.

116. The workstation of claim 114, wherein the operation comprises an operation on a fluid to be delivered to a channel, reservoir and/or cell chamber in the substrate.

117. The workstation of claim 116, wherein the operation comprises delivery of fluid, an agent, a cell, pressure, a voltage, a current, ultrasound, light, and /or a radiofrequency to a region on the substrate.

118. The workstation of claim 114, wherein the operation comprises separation of molecules and/or ions in a fluid to be delivered to a microchannel in the substrate.

119. The workstation of claim 118, wherein the molecules and/or ions are selected from the group consisting of proteins, polypeptides, peptides, nucleic acids, organic molecules, inorganic molecules, carbohydrates, metabolites, positive ions, negative ions, and combinations thereof.

120. The workstation of claim 114, wherein the operation comprises heating or cooling of a fluid to be delivered to a channel in the substrate.

121. The workstation of claim 114, wherein the operation comprises moving the substrate or a component of the substrate.

122. The workstation of claim 121, wherein the component of the substrate comprises a sensor.

123. The workstation of claim 122, wherein the sensor comprises a cell or cell fraction.

124. The workstation of claim 114, wherein the operation comprises exposing a region of the substrate to light from a light source.

125. The workstation of claim 124, wherein the light source is a laser in optical communication with a sensor in a microchannel, reservoir and/or cell chamber of the substrate.

126. The workstation of claim 68, wherein the workstation further comprises a user device for generating and displaying a graphical user interface in response to instructions from the computer program product.

127. The workstation of claim 126, wherein the graphical user interface displays a screen on which at least one substrate property, property of a sensor, parameter of substrate function, and/or parameter of the macroscale device function is displayed.

128. The workstation of claim 126, wherein the graphical user interface displays a screen comprising fields for inputting one or more function parameters.

129. The workstation of claim 128, wherein the function parameters are selected from the group consisting of: arrival of an analyte, agent, and/or cell at a channel outlet, arrival of an analyte, agent, and/or cell at a microchannel inlet, fluid movement through the at least one channel, an electroporation event, an electrophoresis event, a concentration event, a separation event, a mixing event, a recording event, a scanning event, pressure, a change in pressure, fluid velocity, a change in fluid velocity, a parameter of an electric field, and combinations thereof.

130. The workstation of claim 129, wherein the graphical user interface displays a screen providing selectable options for a plurality of different scan modes for scanning a sensor relative a substrate, by moving the sensor, the substrate, the substrate and sensor, and/or by varying pressure at least one microchannel of the substrate.

131. The workstation of claim 68, wherein the data acquisition system comprises a data acquisition program comprising a search function, a data organizing or managing function, a data mining function, a relationship-determining function and/or a data retrieval function.

132. The workstation of claim 68, wherein the data acquisition system further comprises a memory comprising data relating to scanning a sensor across one or more fluid streams of the microfluidic substrate and/or data relating to pressure changes at at least one microchannel of the microfluidic substrate.

133. The workstation of claim 132, wherein the data relating to scanning the sensor comprises data relating to the number of microchannel outlets scanned, the time to complete a scan, pause time intervals at one or more channels, a type of fluid stream delivered by one or more microchannel outlets, pressure in one or more microchannels, fluid velocity in one or more microchannels, and data relating to the sensor response at one or more microchannel outlets.

134. The workstation of claim 68, wherein the workstation further comprises a stage for receiving the substrate which can be moved in one or more of an x-, y-, or z-direction and/or by rotating and/or by tilting.

135. The workstation of claim 68, wherein the workstation further comprises a computer program product for patch clamp data acquisition and analysis.

136. The workstation of claim 134, further comprising one or more mechanisms for controlling the movement of the stage.

137. The workstation of claim 136, wherein the one or more mechanisms comprises one or more joysticks.

138. The workstation of claim 126, wherein the graphical user interface displays a representation of the substrate on a screen of the user interface.

139. The workstations of claim 138, wherein coordinates of the representation are selected and in response to the selecting, an operation at corresponding coordinates on the substrate occurs.

140. A suite of computer program products comprising a computer program product according to any of claims 1-3 and a data acquisition program for patch clamp data acquisition.

141. The suite of computer program products according to claim 140 wherein the data acquisition program comprises computer program code for analyzing patch clamp data.

142. A system comprising:
a first computer program product according to any of claims 1-3;
a second computer program product comprising computer program code for acquiring data relating to properties of a sensor in fluid communication with at least one channel of the microfluidic substrate; and
a data accessing system for accessing the data relating to properties of the sensor and for providing the data to the first computer program product.

143. The system according to claim 142, wherein the system further comprises a microfluidic substrate operably linked thereto and wherein in response to data provided to the first computer program product, instructions from the first computer program product are executed, changing one or more parameters of one or more functions of the microfluidic substrate.

144. The system according to claim 142, wherein the one or more fictions of the microfluidic substrate comprise scanning a sensor relative to an outlet of at least one microchannel in the substrate by moving the sensor, moving the substrate, moving both the sensor and the substrate, and or varying pressure at one or more microchannels of the substrate.

145. A method, comprising
providing a sensor in fluid communication with at least one microchannel of a microfluidic substrate;
providing data to a computer program product for according to any of claims 1-3, wherein in response to the data provided, the computer program product provides instructions to a scanning mechanism to execute one or more scanning functions such that the substrate, the sensor, or the substrate and the sensor move relative to one another, and/or such that pressure is altered in at least one microchannel of the substrate.

146. The method of claim 145, wherein an outlet of at least one microchannel of the substrate delivers a fluid stream which contacts the sensor.

147. The method according to claim 146, wherein the substrate comprises a plurality of microchannels with outlets opening into a sensor chamber containing the sensor and wherein the sensor is exposed to a plurality of fluid streams in a sequence.

148. The method according to claim 147, wherein the sequence is pre-programmed.

149. The method according to claim 145, wherein scanning is continuous.

150. The method according to claim 145, wherein the sensor is paused at one or more channel outlets during a selected time interval.

151. The method according to claim 145, wherein at least one of the fluid streams comprises an agent.

152. The method according to claim 145, wherein the fluid streams provide interdigitating fluid streams of agent and buffer and the sensor is sequentially scanned across the fluid streams.

153. The method according to claim 145, wherein the sensor is stationary and scanning occurs by varying pressure across one or more channel in proximity to the sensor.

154. The method according to claim 147, wherein the sequence is selected based on a response of the sensor.

155. The method of claim 146, wherein the method further comprises measuring a response of the sensor to one or more fluid streams.

156. The method of claim 155, wherein the response comprises a change in an electrical property of the sensor.

157. The method of claim 145, wherein the sensor is a cell or cell fraction.

158. The method of claim 156, wherein the response is measured by measured by patch clamp.

159. The method of claim 155, wherein the response is measured after exposure of the sensor to an electric field.

160. The method of claim 145, wherein the method further comprises the step of entering data relating to one or more properties of the substrate into the display of an interface of a user device in communication with the data processing system, and wherein in response to the entering, the sensor is scanned across the one or more fluid streams and/or pressure is varied at one or more channels.

161. A method for executing one or more functions of a microfluidic substrate comprising executing program code of a computer program product according to any of claims 1-67, wherein the computer program product is operably linked to the microfluidic substrate.
